(19) United States
(12) Patent Application Publication
(10) Pub. No.: US 2026/0263594 A1
(43) Pub. Date: Sep. 10, 2026

(54) METHOD FOR MANUFACTURING RECOMBINANT TRANSFERRIN BINDING PROTEINS AND VACCINE COMPOSITIONS COMRPISING SAME

(71) Applicant: SERUM INSTITUTE OF INDIA PRIVATE LIMITED, Pune Maharashtra (IN)

(72) Inventors: Abhijeet Jagannath KARALE, Pune Maharashtra (IN); Asha Dinesh MALLYA, Pune Maharashtra (IN); Rajeev Mhalasakant DHERE, Pune Maharashtra (IN); Dipen Jagdishbhai SONI, Pune Maharashtra (IN); Sushil Vardhaman PATNI, Pune Maharashtra (IN); Vinay Vijay GAVADE, Pune Maharashtra (IN)

(21) Appl. No.: 19/121,349

(22) PCT Filed: Oct. 11, 2023
(86) PCT No.: PCT/IN2023/050922

§ 371 Date: Apr. 15, 2025

(30) Foreign Application Priority Data
Oct. 18, 2022 (IN) 202221059530

(51) Int. Cl. A61K 39/39; A61K 39/00; A61K 39/09; A61P 37/04; C07K 14/22

(52) U.S. Cl. CPC A61K 39/39; A61K 39/092; A61P 37/04; C07K 14/22; A61K 2039/55516

(57) ABSTRACT

The present disclosure relates to manufacturing transferrin binding proteins. Specifically, the present disclosure relates to a simple, scalable, commercially viable fermentation and purification process for obtaining recombinant transferrin binding protein (rTbp-B) along with high recovery, low impurity/aggregate content, and at the same time retains the integrity of the protein. The method uses a single chromatographic step and does not require tagging of proteins as compared to multiple chromatographic steps used previously and still manages to provide r-Tbp-B with at least 95% purity.

### FIELD

The present disclosure relates to receptor's surface lipoproteins. Particularly, the present disclosure relates to recombinant receptor's surface lipoproteins, more particularly recombinant transferrin binding protein (r-Tbp-B), improved methods of preparing highly purified & stable recombinant transferrin binding proteins and their use as adjuvant for vaccines.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Vaccines are biological preparations used to stimulate the body's immune response against diseases and are capable of imparting active acquired immunity against disease(s). Vaccines stimulate the immune response to recognize a pathogen (a disease-causing organism) or part of a pathogen. When the body is exposed to such pathogens, an immune response is triggered in the body resulting in the destruction of the pathogens. Vaccines may be prophylactic (to prevent or ameliorate the effects of a future infection by a pathogen: such as polio virus, rotavirus, measles), or therapeutic (to fight a disease that has already occurred, such as cancer).

Vaccines may contain whole bacteria or viruses: the bacteria or viruses may be weakened (attenuated) so that they cannot cause disease in healthy people, or killed altogether (inactivated). Many vaccines contain only parts of viruses or bacteria, usually proteins or sugars from the surface. These stimulate the immune system but cannot cause disease. Recent developments in vaccine manufacturing include use of recombinant DNA/RNA technology-based vaccines.

Different types of vaccine include live-attenuated vaccines; inactivated vaccines; subunit, recombinant, polysaccharide-protein conjugate vaccines; toxoid vaccines; mRNA vaccines; and viral vector vaccines.

Vaccination is the most effective method of preventing infectious diseases. Vaccination aids in reduction of morbidity and mortality due to infectious diseases, eradication of infectious diseases, helps in developing herd immunity, reduces secondary infections that complicate vaccine-preventable diseases. The World Health Organization (WHO) reports that licensed vaccines are currently available for twenty-five different preventable infections.

Typically, vaccine contains an active component (the antigen) which generates the protective immune response; and may contain additional components to enhance immune response/efficacy of the vaccine, to stabilize the vaccine and to keep the vaccines safe. These additional components are included in most vaccines and are being used for a long time in billions of doses of vaccine. Each vaccine component serves a specific purpose, and each ingredient is tested in the manufacturing process. All ingredients used in a vaccine composition are stringently tested for safety as these are administered to healthy individuals including infants, children and adults. Vaccines can be administered via oral route, intranasal route, and through needle injections (intramuscular, intradermal, subcutaneous, intravenous, etc).

The antigen may be a small part of the pathogen, like a protein or sugar, or it may be the whole organism in a weakened or inactive form. Vaccines may contain additional components such as stabilizers, preservatives, surfactants, diluents, adjuvants and the like.

An adjuvant improves the immune response to the vaccine, sometimes by keeping the vaccine at the injection site for a little longer or by stimulating local immune cells. Vaccine adjuvants were first described about 80 years ago and have been used to improve immune responses against non-living vaccines since then. Although the role of an adjuvant is to improve the immunogenicity of antigens, they are often included in vaccines to achieve a range of more specific effects. Vaccines that comprise attenuated live organisms or whole inactivated organisms do not generally require adjuvants.

Antibody responses such as bactericidal, virus neutralizing, inactivation may be enhanced by the use of adjuvants. Adjuvants are capable of inducing cell-mediated immunity, e.g. TH1 cytokines (IFN-γ), helps in decreasing the dose of antigen in the vaccine, can decrease the number of doses of vaccine necessary, can overcome competition between antigens in combination vaccines, and enhance immune responses in the young or elderly, who often respond poorly to vaccines. Adjuvants can also improve immune responses in populations where responses to vaccines are typically reduced, such as infants, the elderly and the immunocompromised.

Alum was the first adjuvant licensed for vaccine use and is capable of enhancing immune response to an antigen by activating Th2-mediated cellular mechanisms. It is not able to induce other cellular immune pathways and is believed to be associated with toxicity problems. Particularly Aluminum-containing adjuvants are found in several conjugate vaccines such as Hib conjugate vaccine (Pedvax-Hib and Vaxem-Hib), Pneumococcal conjugate vaccine (Synflorix and Prevenar), Meningococcal conjugate vaccines (MENJUGATE, NEISVAC-C,) and in combination vaccines such as DTP-HB-Hib-IPV, DTPHB-Hib and DTP-Hib, (D, Diphtheria; T, Tetanus; P, Pertussis; HB, Hepatitis B; Hib, Haemophilus influenza type b; IPV, Inactivated Polio Vaccine).

However, there are several drawbacks of using Alum adjuvants for vaccines. For instance, inoculation of aluminum hydroxide-based adjuvant vaccine can cause local adverse reactions such as erythema, subcutaneous nodules, contact hypersensitivity and granuloma. Several studies have concluded that aluminum hydroxide-containing vaccines can lead to local tissue damage with symptoms similar to MMF (macrophagic myofasciitis) when injected intramuscularly. MMF-like transient damages were also observed in experimental animal models that were injected with vaccines with aluminum hydroxide-based adjuvant intramuscularly.

Allergic reactions include another critical adverse drug reaction (ADR) of aluminous adjuvants. Firstly, acidophilic cells could be attracted by adjuvants to the site of injection, which in turn could lead to the increase in total IgE levels. These induce IgE-mediated allergies, which could potentially increase the sensitivity of susceptible individuals.

Low doses of alum may get deposited in kidney leading to renal disorders. Induction of autoimmunity is also associated with vaccine containing alum adjuvants.

Additionally, there is evidence that the inclusion of alum may decrease the thermal stability of some vaccine antigens, which is of critical importance for global pandemic preparedness. Clapp, T et al J. Pharm. Sci. 2011, 100, 388-401.

Alum adjuvants are not able to activate Th-1 dependent cellular mechanisms, and cannot be used in malarial vaccines, tuberculosis, cancer and allergic diseases, where Th1 pathway of activation might be protective.

Another drawback of aluminum hydroxide-based adjuvant is that they cannot be stored frozen. Antigens in vaccines with aluminum hydroxide-based adjuvant are adsorbed and supported by the grid structure of aluminum salt, which is prone to destruction when frozen. Therefore, aluminum hydroxide-based adjuvant vaccines cannot be stored below zero degree Celsius. Peng He et al Hum Vaccin Immunother. 2015 February; 11(2): 477-488.

Other adjuvants used in licensed vaccine include emulsion adjuvant MF59, which is oil in water emulsion of squalene oil, which is a naturally occurring substance found in plants and in a range of animal species, including humans. MF59 is used in licensed Influenza vaccine; Fluad and Fluad Quadrivalent. MF59 has been used in vaccines in Europe since 1997 and in the United States since 2016, has been given to millions of people and has an excellent safety record.

AS01 is an adjuvant suspension of Liposome co-delivery of saponin QS-21 and MPL and is used in herpes zoster (Shingrix) malaria vaccine. AS01 activates TLR4 in innate immune cells and caspase 1 in subcapsular sinus macrophages, induces differentiation of monocytes to DC, and activates NF-κB and production of IFNγ.

AS03 is an Emulsion of Squalene, α-tocopherol, and Tween (polysorbate) 80 used for H1N1 influenza virus. AS03 activates human monocytes and macrophages and induces NF-κB activity and chemokine production.

AS04 (monophosphoryl lipid A (MPL) adsorbed on to alum) was used in U.S. vaccine (Cervarix®) from 2009 for HPV infections. This immune-boosting substance was isolated from the surface of bacteria. MPL activates TLR4 and NF-κB to stimulate antigen presenting cells and innate immune system while alum causes the release of DAMPs and local inflammation.

CpG 1018 is one of the newer adjuvants used in Heplisav-B vaccine. It is made up of cytosine phosphoguanine (CpG) motifs, which is a synthetic form of DNA that mimics bacterial and viral genetic material. When CpG 1018 is included in a vaccine, it increases the body's immune response by Activating TLR9 resulting in a type I interferon response. In pre-licensure clinical trials, adverse events after Heplisav-B were comparable to those observed after another U.S.-licensed, non-adjuvanted hepatitis B vaccine.

MatrixM™ adjuvant is made from saponins derived from the soapbark tree (Quillaja saponaria Molina), along with cholesterol and phospholipids. It is currently used in the Novavax COVID-19 vaccine to stimulate the immune system. On Jul. 19, 2022, the Advisory Committee on Immunization Practices made an interim recommendation for use of the Novavax vaccine in people 18 years and older as a primary 2-dose series vaccination for the prevention of COVID-19.

Other adjuvants, such as virosomes are composed of unilamellar liposomes composed of viral proteins and phospholipids of vaccine target virus and are used in Seasonal flu and hepatitis A vaccine. Virosomes contain PAMPs on the surface that stimulate and activate antigen-presenting cells while also facilitating antigen delivery.

Viral Vectors such as Adenoviruses are also used as adjuvants. Adenovirus carrying mRNA encoding for protein antigen Covid-19. Pathogen-associated molecular patterns (PAMPs) on the surface of adenoviral carriers activate the innate immune system while also facilitating transfection for mRNA vaccines. Current adjuvants enable dose sparing, but the ideal vaccines also need to be cost-effective and require as few doses as possible to mount a rapid and comprehensive vaccination campaign across the globe, including in low-resource settings.

Despite the successes of adjuvants currently included in licensed vaccines, some of the recent adjuvants have not been able to demonstrate the required result(s). CoronaVac (Covid-19 vaccine with alum as adjuvant) provides less robust protection than the other commercially available COVID-19 vaccines, which is only marginally improved with additional doses, and is less effective in older populations. Also, for the outbreak of SARS (severe acute respiratory syndrome) and MERS (Middle East Respiratory Syndrome), studies have demonstrated the safety issue using vaccine adjuvanted with aluminum salts (Honda-Okubo Y et al J Virol. 2015; 89(6):2995-3007, Lokugamage K G et al Vaccine. 2008; 26(6):797-808 & Agrawal A S et al Hum Vaccines Immunother. 2016; 12(9):2351-56). Thus, it is necessary to develop new adjuvants to solve these new challenges.

Malaria vaccine (RTS,S/AS01) adjuvanted with AS01 protects only 36% of children after four doses delivered on an optimized, non-standard schedule. This is an especially large burden relative to the vaccine's value since the protection provided by the vaccine drops to effectively zero within seven years. While additional doses of either RTS,S/AS01 or CoronaVac vaccine may improve their efficacy, given limited global vaccine supply and healthcare resources, this is logistically very challenging.

Toxicity of adjuvants should be balanced against the benefits of incorporating them in vaccine formulations. Depending on the adjuvant components, toxic reactions could be either mild or severe. After injection of vaccine-adjuvant formulations local inflammation, necrosis, granuloma formation was reported at the injection site. Systemically fever, nausea, eosinophilia and allergy reactions can occur due to adjuvant toxicity. Unfortunately, potency of an adjuvant is directly correlating to their toxicity status. Familiar example is the complete Freund adjuvant, a potent adjuvant associated with severe local and systemic toxicities. Therefore, it is important to reduce toxicity of adjuvants, while maximizing their potency.

Some of the biggest challenges in executing rapid and comprehensive vaccination campaigns in response to pandemics are quickly producing a sufficient number of doses for global use, keeping cost low to ensure accessibility, and maintaining safety-all of which could potentially be addressed by adjuvants. Thus, new antigen-agnostic adjuvants that leverage our growing ability to manipulate the immune system in a way that is both safe and confers robust immunity can help to improve preparedness for future pandemics. Advanced off-the-shelf adjuvants that have already been evaluated for safety and can be quickly tested with antigens against an emerging pathogen would allow researchers to improve the efficacy, development timeline, price, and supply of future vaccines.

Although we cannot predict precisely what pathogen will cause the next pandemic, we can rationally engineer and characterize adjuvants to promote specific immune responses across a broad array of antigens so that when a new pathogen and immunity-conferring antigen are identified, we can rapidly develop a potent vaccine. One potentially valuable approach to preparing for the next pandemic would be to create a library of well-characterized, “ready-to-use” adjuvant platforms. Ideally, these adjuvants would have previously demonstrated the ability to promote a robust and protective immune response for a diverse set of antigens so that pandemic antigens can be rapidly tested with promising candidates to determine the most effective vaccine formulation.

Though some of the recent adjuvants are capable of eliciting cellular immunity, they fail to produce strong cellular immune response when tested in humans.

Applicant has specifically found that although Alum adjuvants have been conventionally used and found effective for polysaccharide-protein conjugate vaccines, for few polysaccharide-protein conjugate vaccines, surprisingly alum has resulted in low immune responses (for instance Streptococcus pneumoniae serotype 3 polysaccharide-CRM197 conjugate, Streptococcus pneumoniae serotype 19A polysaccharide-CRM197 conjugate). Further for polysaccharide-protein conjugate vaccines, apart from Alum, other alternative adjuvants have not been effective, they fail due to complex manufacturing processes, poor stability. Therefore, conventional alum adjuvant is not an ideal choice for a vaccine antigen with poor immunogenicity.

The ideal new adjuvants should have a broad-spectrum of safety, should exhibit either equivalent or improved immune response to vaccine antigens when compared to responses derived by using conventional Alum and previously known alternative adjuvants, should be economical from manufacturing perspective, ease of production and use, should be stable, should have compatibility with antigen and should effectively activate humoral and cellular immune responses with no adverse reactions. Adjuvants that are being developed must tackle these concerns to make global vaccination more feasible and accessible worldwide. Therefore, there is still a need to develop adjuvants that are capable of eliciting strong immune response, are safe and non-toxic, and can be easily manufactured on a large scale.

Tbp-B is an outer membrane lipoprotein presumably attached to the outer membrane through an N-linked terminal lipid anchor. Tbp-B, along with Tbp-A, is expressed by Neisseria meningitidis to overcome the lack of bioavailable iron within the host by acquiring its iron directly from human transferrin. Based on the antigenic and genomic features of Tbp-B, N. meningitidis isolates can be classified into two major families: isotype I and isotype II. The two Tbp-B isotypes are not equally represented among N. meningitidis clonal complexes as isotype I is mainly restricted to the ST11 complex while isotype II is widely represented in all other major complexes responsible for meningococcal infection.

Ian C. Boulton et al/Centre for Applied Microbiology and Research, UK (1998) & others have reported purifying Tbp-B using at least 2 sequential chromatographic steps. Following are few drawbacks of multiple chromatography steps (Affinity; IEX, SEC) for purification of recombinantly expressed E. coli proteins, same may have an impact on stability/structure/integrity of protein of interest 1) Resultant recombinant proteins (from tag-based purification) reported previously are recovered in non-native conformation. 2) SEC, IEX and HIC are often coupled downstream the affinity purification procedure for sample refining. SEC disadvantage—The dilution of the protein sample during separation, which may alter equilibria between oligomeric species, while a concentration step may be required for downstream applications, which may induce protein precipitation. 3) On a commercial scale, due to the multistep process, the final yield decreases to a great extent. 4) Tag based Affinity purification suffers from several disadvantages the fact of being incapable of solving problems of protein heterogeneity, as charge and size variability. If tags are not removed prior to further analysis of the peptide, even a small tag, such as a 6×His-tag, can greatly influence the protein structure and aggregation propensity (Jia, L., Wang, W. et al 2019; Amyloidogenicity and Cytotoxicity of a Recombinant C-Terminal His 6-Tagged Aβ 1-42). Moreover, removal of the tag requires the addition of a cleavage recognition site and additional purification steps which lead to loss of protein, increased time of protein handling, and therefore formation of aggregated species. Further while adopting tagged based protein purification extensive studies are required for selecting the compatible tag type, size and location which makes purification process complex. Thus, the purification processes described previously for E coli expressed recombinant proteins are complicated, none of the earlier processes reveal a basic, prudent and financially practical strategy for the production of Tbp-B which could ensure the consistent production of stable product at industrial scale.

Previously, use of glass homogenizer (or Emulsiflex, High pressure homogenizer etc) for recovery of recombinant proteins in E coli has been reported. High pressure homogenizers can be expensive to purchase and maintain, requiring specialized training and knowledge, may not be suitable for certain types of samples, such as heat-sensitive samples or samples that are prone to oxidation. However, the use of a French press to lyse bacterial cultures is useful for large volume samples, but the limited availability of such equipment and high cost are often prohibitive. In addition, the use of a French press is time consuming and not well suited for small samples or high throughput sample processing (Goeddel et al 1979, Schumacher et al. 1986). French press mediates extremely severe lysis that further disrupts and releases membrane proteins that can interfere with the purification of target proteins. This method often leads to degradation and also suffers from populations of unlysed or incompletely lysed cells. Further the capital cost of the bead mill or high-pressure homogenizers used in this work and reported by others to produce CPFS extract is approximately $10,000 to $40,000 compared with the commonly available sonicator, which costs about $4000.

Hence a there is need for a simplified process for preparing highly pure & stable recombinant transferrin binding proteins which involves fewer steps and gives higher yields in a short time.

### OBJECTS

Some of the objects of the present disclosure, which at least one embodiment herein satisfies, are as follows:

An object of the present disclosure is to ameliorate one or more problems of the prior art or to at least provide a useful alternative.

Another object of the present disclosure is to provide recombinant transferrin binding proteins (rTbp-B) to be used in vaccine compositions.

Still another object of the present disclosure is to provide an improved method for manufacturing and purifying recombinant transferrin binding protein (rTbp-B).

Yet another object of the present disclosure is to manufacture recombinant transferrin binding proteins (rTbp-B) having high purity and stability.

Still another object of the present disclosure is to manufacture recombinant transferrin binding proteins that can be used as adjuvant or immunomodulator for vaccines/antigens or therapeutics to provide enhanced/desired immune response.

Another object of the present disclosure is to provide recombinant transferrin binding proteins (rTbp-B) capable of use as carrier protein in polysaccharide-protein conjugate vaccine composition providing improved immune response as compared to polysaccharide-protein conjugate vaccine prepared using conventional carrier proteins.

Yet another object of the present disclosure is to provide recombinant transferrin binding (rTbp-B) proteins capable of use as antigen.

Other objects and advantages of the present disclosure will be more apparent from the following description, which is not intended to limit the scope of the present disclosure.

### SUMMARY

The present disclosure provides a method for manufacturing a recombinant transferrin binding protein (rTbp-B) comprising a single chromatography step. The method comprises:
a. providing a cloning vector comprising a gene sequence encoding recombinant transferrin binding protein B;
b. transferring the cloning vector into a host system and growing the host system;
c. inducing the host system using an inducing agent at a final concentration in the range of 0.1 mM to 2.0 mM, preferably 0.1 mM to 1 mM;
d. harvesting/collecting the host system cells and subjecting it to centrifugation to collect the pellet containing inclusion bodies, wherein the transferrin binding protein is contained in the inclusion bodies;
e. washing the inclusion bodies, followed by solubilizing the inclusion bodies to obtain crude recombinant transferrin binding protein; and
f. subjecting the crude recombinant transferrin binding protein to an ion exchange chromatography to obtain the recombinant transferrin binding protein,
wherein the recombinant transferrin binding protein has >95% purity.

The method of present disclosure provides a simple, scalable, commercially viable fermentation and purification (using improved fermentation medium, optimized induction and improved cell lysis by optimized sonication parameters) process for obtaining recombinant transferrin binding protein (rTbp-B) along with high recovery, low impurity/aggregate content, and at the same time retains the integrity of the protein. The method uses a single chromatographic step (anion exchange chromatography) as compared to multiple chromatographic steps (affinity Chromatography, ion exchange and hydrophobic interaction chromatography) used previously and still manages to provide r-Tbp-B with at least 95% purity. One of the advantages of the present method is that there is no involvement of tagging proteins. Further, the r-Tbp-B of the present disclosure is stable at 2-8° C., and 25° C. and vaccine compositions containing r-Tbp-B is capable of slowly releasing antigen, allowing the antigen to be exposed to immune system for longer periods of time, resulting in higher B and T cell response.

### BRIEF DESCRIPTION OF THE ACCOMPANYING DRAWING

The present disclosure will now be described with the help of the accompanying drawing, in which:

FIG. 1 illustrates the comparison of fractions obtained by CNBr activated resin and anion exchange chromatography method of the present disclosure;

FIG. 2 illustrates the comparison of fractions obtained by tagging proteins and without tagging proteins;

FIG. 3 illustrates the SDS-PAGE for anion exchange chromatography elutes collected at different salt gradients;

FIG. 4 illustrates the Western Blotting analysis of the elutes obtained by the method of the present disclosure;

FIG. 5 illustrates the molecular weight of purified transferrin binding protein by SEC-MALS;

FIG. 6 illustrates the peptide fingerprinting to determine the sequence homology of purified transferrin binding protein;

FIG. 7 illustrates the Mass Spectrometry analysis of purified transferrin binding protein;

FIGS. 8a-8c illustrate the Surface Plasmon resonance (SPR) analysis of purified transferrin binding protein;

FIG. 9 illustrates the densitometry analysis of purified transferrin binding protein;

FIGS. 10a-10d illustrate the stability studies of purified transferring binding protein as adjuvant;

FIGS. 11a-11r illustrate the stability studies of purified transferring binding protein as carrier protein, wherein FIGS. 11a-11i illustrate the SEC-MALS analysis and FIGS. 11j-11r illustrate the SEC-HPLC analysis; and

FIGS. 12a-12c illustrate the adjuvant activity of transferrin binding protein of the present disclosure.

### DESCRIPTION

Although the present disclosure may be susceptible to different embodiments, certain embodiments are shown in the drawing and following detailed discussion, with the understanding that the present disclosure can be considered an exemplification of the principles of the disclosure and is not intended to limit the scope of disclosure to that which is illustrated and disclosed in this description.

Embodiments are provided so as to thoroughly and fully convey the scope of the present disclosure to the person skilled in the art. Numerous details are set forth, relating to specific components, and processes, to provide a complete understanding of embodiments of the present disclosure. It will be apparent to the person skilled in the art that the details provided in the embodiments should not be construed to limit the scope of the present disclosure. In some embodiments, well-known composition, well-known processes, and well-known techniques are not described in detail.

The terminology used, in the present disclosure, is only for the purpose of explaining a particular embodiment and such terminology shall not be considered to limit the scope of the present disclosure. As used in the present disclosure, the forms “a”, “an”, and “the” may be intended to include the plural forms as well, unless the context clearly suggests otherwise.

The terms “comprise”, “comprising”, “including”, and “having” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the process of the present disclosure is not to be construed as necessarily requiring their performance as described or illustrated. It is also to be understood that additional or alternative steps may be employed.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention.

The present disclosure envisages a method for manufacturing recombinant transferrin binding protein (r-Tbp-B). The method comprises cloning and expression of recombinant transferrin binding protein as inclusion bodies in an expression host system; washing of Inclusion Bodies (IB) containing the recombinant transferrin binding protein; using single step chromatography for purification of r-Tbp-B. The method does not involve tagging of transferrin binding proteins during purification; which reduces the time duration required for the entire process, is less tedious and laborious, but at the same time results in r-Tbp-B having >95% purity.

The terms recombinant transferrin binding protein B, r-Tbp-B, rTbp-B, Tbp-B are interchangeably used throughout the specification.

In an aspect of the present disclosure, there is provided a method for manufacturing recombinant transferrin binding protein (r-Tbp-B). The method comprises a single chromatography step. The method in accordance with the present disclosure comprises the following steps:
providing a cloning vector comprising a gene sequence encoding recombinant transferrin binding protein B;
transferring the cloning vector into a host system and growing the host system;
inducing the host system using an inducing agent at a final concentration in the range of 0.1 mM to 2.0 mM;
harvesting/collecting the host system cells and subjecting it to centrifugation to collect the pellet containing inclusion bodies, wherein transferrin binding protein is contained in the inclusion bodies;
washing the inclusion bodies, followed by solubilizing the inclusion bodies to obtain crude recombinant transferrin binding protein; and
subjecting the crude recombinant transferrin binding protein to anion exchange chromatography to obtain the recombinant transferrin binding protein.

The r-Tbp-B obtained by the method of present disclosure has a >95% purity.

In another embodiment of the present disclosure, the recombinant transferrin binding protein is obtained from bacteria selected from the group consisting of, but not limited to, Streptococcus spp, such as Group A Streptococcus, Group B Streptococcus (group Ia, Ib, II, III, IV, V, VI, VII, VII, VIII, and IX); Streptococcus pneumoniae, Streptococcus pyogenes; Streptococcus agalactiae; Streptococcus viridans; Salmonella spp, such as, Salmonella typhi; Salmonella paratyphi; Salmonella enteritidis; Salmonella typhimurium; Shigella spp, such as Shigella sonnei, Shigella flexneri, Shigella dysenteriae; Shigella boydii; E. coli; Neisseria meningitidis (serotypes such as A, B, C, D, E29, H, I, K, L, M, W135, X, Y, Z, etc); Neisseria gonorrhoeae; Haemophilus influenzae; Haemophilus pneumonia; Helicobacter pylori; Chlamydia pneumoniae; Chlamydia trachomatis; Ureaplasma urealyticum; Mycoplasma pneumoniae; Staphylococcus spp, such as Staphylococcus aureus, Staphylococcus aureus type 5, Staphylococcus aureus type 8; Enterococcus faecalis; Enterococcus faecium; Bacillus anthracis; Vibrio cholerae; Pasteurella pestis; Pseudomonas aeruginosa; Campylobacter spp. such as jejuni; Clostridium spp, such as Clostridium difficile; Mycobacterium spp, such as Mycobacterium tuberculosis; Moraxella catarrhalis; Klebsiella pneumoniae; Treponema spp.; Borrelia spp.; Borrelia burgdorferi; Leptospira spp.; Hemophilus ducreyi; Corynebacterium diphtheria; Bordetella pertussis; Bordetella parapertussis; Bordetella bronchiseptica; Ehrlichia spp.; and Rickettsia spp.

In an embodiment of the present disclosure, the recombinant transferrin binding protein may be derived from a Neisseria meningitidis selected from the serotypes consisting of, but not limited to A, B, B16, B6, C, D, E29, H, I, K, K454 L, M, W135, X, Y, and Z.

The Neisserial transferrin receptor complex is composed of two surfaces exposed, iron repressible proteins-transferrin binding protein A (Tbp-A), and transferrin binding protein B (Tbp-B). Tbp-A is an integral outer membrane protein, with sequence homology to the TonB-dependent family of outer membrane proteins. Tbp-B is a lipidated surface exposed protein with a variable molecular weight between 60 to 90 kDa. Tbp-B is considered to be an outer membrane protein that is anchored to the membrane via the lipidated N-terminal part of the protein. Tbp-B is able to discriminate between iron loaded transferrin and iron free apo-transferrin. Thus, Tbp-B is thought to increase the efficiency of this receptor complex by discriminating between iron-loaded and iron-depleted transferrin.

Transferrin binding protein B (Tbp-B), a recombinant version of the receptor's surface lipoprotein component, can be easily synthesized in large quantities in the cytoplasm of Escherichia coli and is ideal for commercial vaccine production. Transferrin binding protein A (Tbp-A), an integral outer membrane protein, is generated at low levels in the outer membrane and requires detergents for solubilization and stability, methods that are not suitable for commercial usage.

In an embodiment of the present disclosure, the recombinant transferrin binding protein is cloned in a cloning vector selected from the group comprising pET-30a (+), pET 23b, pET 28a, pET 28b, pET 23a, and pET 22a.

In accordance with the embodiments of the present disclosure, the cloning vector comprises an antibiotic marker selected from the group consisting of kanamycin, streptomycin, and neomycin. In a preferred embodiment, the antibiotic marker is kanamycin.

The cloning vector is transferred into a host system and the host system is grown under suitable conditions.

In an embodiment of the present disclosure, the recombinant transferrin binding protein may be expressed in a host system selected from the group comprising prokaryotic and eukaryotic expression host systems, such as Bacillus brevis, Bacillus megaterium, Bacillus subtilis, Caulobacter crescentus, Escherichia coli (such as Escherichia coli BL21, E coli Cell lines (BL21(DE3); BL21Star(DE3); Origami(DE3); BL21(DE3)pLysS; BL21-CodonPlus(DE3)-RIPL; Rosetta(DE3); C41(DE3)/C43(DE3); Lemo21(DE3); Shuffle; ATCC Deposit No. PTA-126975) used for recombinant expression of proteins JM109, E. coli K12), yeast (such as Saccharomyces cerevisiae), insect (such as Spodoptera frugiperda SF9 or SF21), baculovirus/insect cells and mammalian cell cultures (such as HEK293 and CHO).

The host system is grown in a medium (such as Luria-Bertani Broth) containing an antibiotic, such as kanamycin, streptomycin, and neomycin. The concentration of the antibiotic is in the range of 10 μg/ml to 50 μg/ml. The temperature is in the range of 36° C. to 38° C. and the agitation is in the range of 150 RPM to 300 RPM. The cells are typically allowed to grow till an OD_{590 nm} of 0.5 to 0.7 is reached.

The host system is induced for optimum protein expression. In accordance with the embodiments of the present disclosure, the inducing agent include, but is not limited to, lactose, isopropyl β-D-1-thiogalactopyranoside (IPTG), and functionally equivalent analogues.

In a preferred embodiment, the inducing agent is isopropyl β-D-1-thiogalactopyranoside (IPTG).

Typically, the final concentration of inducing agent is in the range of 0.1 mM to 2.0 mM (preferably 0.1 to 1 mM). The cells are allowed to grow at a temperature in the range of 36° C. to 38° C. and the agitation in the range of 150 RPM to 300 RPM. In an embodiment, the cells are allowed to grow for a time period of 2 hours to 10 hours.

The induced cells are allowed to grow and are subsequently harvested/collected and is centrifuged at 5000 RPM to 15000 RPM for 5 minutes to 20 minutes at 2° C. to 8° C. to collect the pellet. The pellet contains the inclusion bodies (IB) in insoluble form and the transferrin binding protein is present in the IB.

The insoluble inclusion bodies are washed using the following protocol:
lysis buffer (10 mM to 100 mM Tris-Cl, 10 mM to 100 mM EDTA, 5% to 50% sucrose, pH 7.0 to 9.0) is added to the pellet;
lysozyme (1 mg/ml to 10 mg/ml) is added and thoroughly mixed, followed by incubation at 36° C. to 38° C., agitation at 150 RPM to 300 RPM for a time period of 30 minutes to 180 minutes;
sonicating the solution at 50 Amp to 200 Amp for a time period of 1 minute to 10 minutes. In an embodiment, the sonication is carried out in an ON/OFF mode;
centrifuging the solution at 5000 RPM to 20000 RPM for 5 minutes to 20 minutes at 2° C. to 8° C.;
decanting the supernatant and collecting the pellet;
adding wash buffer (0.1 M to 2 M NaCl, 0.5% to 10% Triton X-100) to the pellet, mixing thoroughly and centrifuging the solution (at 5000 RPM to 20000 RPM for 5 minutes to 20 minutes at 2° C. to 8° C.);
decanting the supernatant and collecting the pellet;
adding NaCl (0.1 M to 2 M) to the pellet, mixing thoroughly and centrifuging the solution (at 5000 RPM to 20000 RPM for 5 minutes to 20 minutes at 2° C. to 8° C.);
decanting the supernatant and collecting the pellet;
adding distilled water to the pellet, mixing thoroughly and centrifuging the solution (at 5000 RPM to 20000 RPM for 5 minutes to 20 minutes at 2° C. to 8° C.);
decanting the supernatant and collecting the pellet;
washing the pellet with distilled water/Milli-Q, mixing thoroughly with distilled water or Milli-Q water and centrifuging the solution (at 5000 RPM to 20000 RPM for 5 minutes to 20 minutes at 2° C. to 8° C.);
adding Tris-Cl buffer (10 mM to 100 mM Tris, 200 mM to 700 mM NaCl; pH 6.0 to 9.0) and incubating at 36° C. to 38° C. for a time period of 30 minutes to 180 minutes; and
centrifuging the solution at 5000 RPM to 20000 RPM for 5 minutes to 30 minutes at 2° C. to 8° C. to separate the supernatant and the pellet.

The supernatant contains the soluble fraction and the insoluble fraction (in the form of pellet). The pellet contains the insoluble inclusion bodies and the soluble fraction contains the contaminants, such as host cell protein (HCP), media components and the like. Soluble fraction may also contain minute amount of protein of interest along with host cell DNA and host cell protein (contaminants). Majority of protein (r-Tbp-B) is present in pellet in the form of IB's which are further taken for purification. The insoluble IBs are solubilized before further downstream processing. The soluble and insoluble fractions obtained are analysed by SDS-PAGE to determine the amount of r-Tbp-B present.

The pellet containing insoluble IBs is dissolved in sonication buffer and sonicated at 50 Amp to 200 Amp for a time period of 30 seconds to 1200 seconds. Preferably, the sonication is carried out at 5 Amp to 80 Amp for a time period of 1 minute to 20 minutes. In an exemplary embodiment, the sonication is carried out at 70 Amp for 5 minutes. (1 to 10 mins already claimed) In an embodiment, the sonication is carried out in an ON/OFF mode. This is followed by addition of dispersion buffer. The majority of the r-Tbp-B is present in the IBs, which is insoluble. The IBs were solubilized by addition of urea (5 M to 15 M) (preferably 4-8M). The urea is diluted and completely removed in the subsequent steps.

In an embodiment, 10 kDa cut-off dialysis cassettes is used to remove urea and other small moieties. Cut-off membrane sufficiently removes urea which has molar mass of 60.0 g/mol. Chaperone and other chaotropic agents (Guanidine hydrochloride, thiourea, SDS etc.) can be alternatively used for solubilization followed by refolding.

In an embodiment, the sonication buffer comprises Tris-Cl (50 mM to 200 mM) and glycine (10 mM to 100 mM) and has a pH in the range of 7.0 to 9.0. In another embodiment, the dispersion buffer comprises Tris-Cl (20 mM to 200 mM), glycine (10 mM to 100 mM) and urea (5 M to 15 M) and has a pH in the range of 7.0 to 9.0.

In accordance with the embodiments of the present disclosure, the sonication may be carried in a sonicator selected from Q Sonica Q500 Sonicator, XL-2020 Sonicator, homogenizer, micro fluidizer, French press/X-mesh. In accordance with the embodiments of the present disclosure non-mechanical methods, such as decompression, osmotic shock, thermolysis, freeze-thaw can also be used.

In accordance with the embodiments of the present disclosure, the inclusion bodies present in the dispersion buffer is purified by ion exchange chromatography selected from anion exchange chromatography and cation exchange chromatography. Preferably, anion exchange chromatography is used for purification of inclusion bodies. Said Anion exchange could be either a Strong or weak anion exchange.

In one embodiment, Anion Exchange matrices used as single step provide desired yield/recovery & low impurity (HCP) i.e. <5% of impurities.

In an embodiment of the present disclosure, the single step chromatography is carried out by a multimodal resin having anion-exchange.

The anion exchange chromatography resin is selected from the group comprising methacrylate polymer; sepharose; diethylaminoethyl cellulose; crosslinked agarose beads; ceramic beads filled with a functionalized hydrophilic gel; methacrylate with trimethylammoniumethyl ligands; highly cross-linked cellulose with dextran scaffold; polystyrene/divinyl benzene polymer matrix; agarose matrix; hydrophilic polyvinyl ether base matrix; hydroxylated methacrylic polymer; sephadex base matrix; hydrophilic polymeric support.

Preferably, said anion exchange chromatography resin is selected from the group comprising Macro-Prep® High Q Media (Bio-Rad) anion exchange resin, HiTrap Q HP ion exchange column, DEAE-cellulose, Q-sepharose, Macro-Prep Q, Q-HyperD, Fractogel EMD-TMAE 650, Cellufine Max Q-r, Source 30Q, Source 15Q, DEAE Sepharose, MonoQ, Capto Q, Eshmino Q, Gigacap Q 650M, Nuvia-Q, Cellufine Q-h, MiniQ, DEAE Sepharose Fast Flow, Q Sepharose high Performance, QAE SEPHADEX™ POROS® HQ 50, POROS® PI 50, POROS® D, Mustang Q, Q Sepharose® FF (QSFF),FAST Q SEPHAROSE™ (GE Healthcare), UNOsphere Q, Macro-Prep DEAE from Biorad, Ceramic HyperD Q, ceramic HyperD DEAE, Toyopearl SuperQ-650S, 650M and 650C, QAE-550C and 650S, DEAE-650M, mixed mode chromatography involving Anion exchange, Capto Adhere (anion exchange and hydrophobic interactions).

In an embodiment of the present disclosure, the pre-treatment of the inclusion bodies in dispersion buffer is carried out before the chromatography step.

In an embodiment, the pre-treatment of inclusion bodies (IB) in dispersion buffer includes, but is not limited to, dialysis. The IB in dispersion buffer may be subjected to dialysis before the chromatography step. The IBs in dispersion buffer may be dialyzed overnight (at 2° C. to 8° C.) with more than 2 exchanges of buffer (10 mM to 100 mM Tris, 50 mM to 200 mM NaCl, pH 7.0 to 9.0). This allows protein refolding. Protein linearization, in which a protein's di-sulfide bond breaks free, is made possible by urea treatment. Dialysis with a 10 kDa cut-off membrane allows for protein refolding with the dual benefits of removing urea and other contaminating small molecules, as well as concentrating protein.

The dialyzed sample is loaded on to a suitable resin having a pre-determined column volume. Prior to loading the dialyzed sample on to the resin, the column is equilibrated using an equilibration buffer.

In accordance with the embodiments of the present disclosure, the resin may be quaternary amine functional groups attached to methacrylate copolymer bead (such as. Macro-Prep® High Q Media (Bio-Rad)). In an embodiment of the present disclosure, the column is syringe column having a bed volume in the range of 2 ml to 15 ml. The equilibration buffer is composed of 20 mM to 100 mM Tris-Cl and pH 7.5 to 9.5. In an embodiment, the column is equilibrated using more than 8 column volume of equilibration buffer. Any other suitable column may be used for the purification of inclusion bodies.

The flow-through sample (unreacted) is collected and is subjected to washing using a column wash buffer. The column wash buffer is composed of 20 mM to 100 mM Tris-Cl and pH 7.5 to 9.5. In an embodiment, the column is washed using more than 5 column volume of column wash buffer.

Elution of the bound sample may be carried out using elution buffers. In accordance with the embodiments of the present disclosure more than one elution buffer may be used. The elution buffer comprises Tris-Cl, NaCl and pH 7.5 to 9.5. Other known elution buffers, such as phosphate buffer may also be used.

The purified r-Tbp-B obtained by the method of present disclosure has a purity of >95%. The method of the present disclosure uses a single chromatography step for purification of r-Tbp-B after cytoplasmic expression in Escherichia coli, compared to known methods wherein multiple chromatographic methods were used which was tedious, labor intensive and time consuming.

The present disclosure provides purified r-Tbp-B having an amino acid sequence having at least 60%, 70%, 80%, 90%, 95%, 97%, 98%, or 99% homology with recombinantly purified Tbp-B (from E. coli) obtained by the method of the present disclosure.

The recombinant transferrin binding protein obtained by the method of the present disclosure is at least 90% identical to SEQ ID NO: 1.

The r-Tbp-B obtained by the method of the present disclosure provides optimum yield and purity. In an embodiment, the yield of r-Tbp-B may be more than 20%, preferably more than 30%. The purity of r-Tbp-B obtained by the method of the present disclosure is not less than 95%, wherein the sum of the impurities, such as aggregate is not greater than 5%. The molecular weight of the r-Tbp-B obtained by the method of the present disclosure may be in the range of 20 kDa to 100 kDa (SEC-MALS). In an embodiment, the molecular weight of the r-Tbp-B obtained by the method of the present disclosure may be less than 70 kDa (SEC-MALS). In a preferred embodiment, the molecular weight of the r-Tbp-B obtained by the method of the present disclosure may be 60 kDa to 70 kDa, preferably 62 kDa to 67 kDa, most preferably 65 kDa (SEC-MALS).

In accordance with the embodiments of the present disclosure, the purified r-Tbp-B obtained may be used in a vaccine composition.

In accordance with the embodiments of the present disclosure, the concentration of r-Tbp-B in a vaccine composition may be in the range of 1 μg to 100 μg, preferably in the range of 5 μg to 70 μg, more preferably in the range of 10 μg to 60 μg.

In an embodiment, r-Tbp-B when used as an adjuvant results in dose sparing of antigen. In accordance with the embodiments of the present disclosure, the vaccine composition comprising r-Tbp-B may be administrated via oral route, intranasal route, and through needle injections (intramuscular, intradermal, subcutaneous, intravenous, etc).

In an embodiment of the present disclosure, the purified r-Tbp-B may be present as an active component in the vaccine composition. The purified r-Tbp-B may be used an antigen to generate protective immune response.

The r-Tbp-B may be present as either standalone or in combination with other antigens or as fusion protein with other Neisserial/pneumococcal proteins in vaccine composition(s).

In an embodiment, r-Tbp-B is used along with r-Tbp-A in vaccine compositions(s).

In another embodiment, r-Tbp-B provides cross protection against all Neisseria strains (Neisseria meningitidis, Neisseria gonorrhoea, etc).

In an embodiment, the vaccine composition comprises a therapeutically effective amount of at least one transferrin binding protein and pharmaceutically acceptable excipients.

In another embodiment of the present disclosure, the purified r-Tbp-B may be present as an additional (additive/excipient) component of the vaccine composition.

In an embodiment of the present disclosure, the purified r-Tbp-B may be present as an adjuvant in a vaccine composition.

In an embodiment of the present disclosure, purified r-Tbp-B may be used as a single adjuvant in a vaccine composition.

In another embodiment of the present disclosure, purified r-Tbp-B may be used as an adjuvant along with other adjuvants (co-adjuvant) in a vaccine composition.

In another embodiment of the present disclosure, the purified r-Tbp-B may be present as a carrier protein in a polysaccharide protein conjugate vaccine composition.

In an embodiment, the vaccine composition comprises
at least one polysaccharide-carrier protein conjugate;
recombinant transferrin binding protein as a carrier protein; and
pharmaceutically acceptable excipients.

In an embodiment, Pnu Type 3-Tbp-B conjugate shows increased immune response by at least 2-fold (IgG) as compared to Pnu Type 3-CRM conjugate. Similarly, for Pnu Type 19A-Tbp-B conjugate, the immune response increases by at least 2-fold (IgG) and at least 7-fold (OPA) as compared to Pnu Type 19A-CRM conjugate.

In still another embodiment of the present disclosure, the purified r-Tbp-B may be present as an additive/excipient which aids in stability, safety and/or efficacy of the vaccine composition.

In an aspect of the present disclosure, there is provided a vaccine composition comprising an antigen and r-Tbp-B as an adjuvant.

In an embodiment, the vaccine composition comprises:
at least one polysaccharide-carrier protein conjugate;
recombinant transferrin binding protein as an adjuvant; and
pharmaceutically acceptable excipients.

The antigen is a protein or fragment thereof, a nucleic acid, a virus, a pseudovirus, a bacterium, or a parasite The bacterial antigen may be selected from the group consisting of, but not limited to, Streptococcus spp, such as Group A Streptococcus, Group B Streptococcus (group Ia, Ib, II, III, IV, V, VI, VII, VII, VIII, and IX); Streptococcus pneumoniae, Streptococcus pyogenes; Streptococcus agalactiae; Streptococcus viridans; Salmonella spp, such as, Salmonella typhi; Salmonella paratyphi; Salmonella enteritidis; Salmonella typhimurium; Shigella spp, such as Shigella sonnei, Shigella flexneri, Shigella dysenteriae; Shigella boydii; E. coli; Neisseria meningitidis (serotypes such as A, B, C, D, E29, H, I, K, L, M, W135, X, Y, Z, etc); Neisseria gonorrhoeae; Haemophilus influenzae; Haemophilus pneumonia; Helicobacter pylori; Chlamydia pneumoniae; Chlamydia trachomatis; Ureaplasma urealyticum; Mycoplasma pneumoniae; Staphylococcus spp, such as Staphylococcus aureus, Staphylococcus aureus type 5, Staphylococcus aureus type 8; Enterococcus faecalis; Enterococcus faecium; Bacillus anthracis; Vibrio cholerae; Pasteurella pestis; Pseudomonas aeruginosa; Campylobacter spp. such as jejuni; Clostridium spp, such as Clostridium difficile; Mycobacterium spp, such as Mycobacterium tuberculosis; Moraxella catarrhalis; Klebsiella pneumoniae; Treponema spp.; Borrelia spp.; Borrelia burgdorferi; Leptospira spp.; Hemophilus ducreyi; Corynebacterium diphtheria; Bordetella pertussis; Bordetella parapertussis; Bordetella bronchiseptica; Ehrlichia spp.; and Rickettsia spp.

In an embodiment, the antigen may be derived from Streptococcus pneumoniae serotype selected from the group consisting of, but not limited to, 1, 2, 3, 4, 5, 6, 6A, 6B, 6C, 6D, 6E, 6G, 6H, 7A, 7B, 7C, 7F, 8, 9A, 9L, 9F, 9N, 9V, 10F, 10B, 10C, 10A, 11A, 11F, 11B, 11C, 11D, 11E, 12A, 12B, 12F, 13, 14, 15A, 15C, 15B, 15F, 16A, 16F, 17A, 17F, 18, 18C, 18F, 18A, 18B, 19A, 19B, 19C, 19F, 20, 20A, 20B, 21, 22A, 22F, 23A, 23B, 23F, 24A, 24B, 24F, 25F, 25A, 27, 28F, 28A, 29, 31, 32A, 32F, 33A, 33C, 33D, 33E, 33F, 33B, 34, 35A, 35B, 35C, 35F, 36, 37, 38, 39, 40, 41F, 41A, 42, 43, 44, 45, 46, 47F, 47A, and 48.

In another embodiment of the present disclosure, the antigen may be derived from Neisseria meningitidis serogroup selected from the group consisting of, but not limited to, A, C, W, Y and X. In an embodiment, the antigen may be derived from N. meningitidis serotype X strains selected from M9601, M9592, M9591, 247X, M9554, M8210 and M2526, 5967 strain (ST 750). In an embodiment, the antigen may be derived from N. meningitidis serotype C strain C11 (60E).

In accordance with the embodiments of the present disclosure, the viral antigen may be selected from the group comprising poxvirus (e.g. orthopoxviruses; avipoxviruses), morbillivirus (e.g. measles), mumps virus, rubella virus, alphavirus (e.g. sendai virus, sindbis virus and semliki forest virus (SFV), ross river virus, encephalitis virus, flavivirus (e.g. yellow fever virus, dengue virus, Japanese encephalitis (JE) virus, a chimeric dengue virus (yellow fever-dengue) virus, a chimeric YF-WN (yellow fever-West Nile virus) virus, a chimeric YF-JE (yellow fever-Japanese encephalitis) virus, Kunjin virus, West Nile (WN) virus, tick-borne encephalitis (TBE) virus, St. Louis encephalitis virus, Murray Valley encephalitis virus, Zika virus), rhabdovirus (e.g. vesicular stomatitis virus (VSV)), retrovirus (e.g. RNA tumor viruses), adenovirus (e.g. human adenovirus, bovine adenovirus, a canine adenovirus, a non-human primate adenovirus, a chicken adenovirus, or a porcine or swine adenovirus), adeno-associated viruses, influenza virus type A (HINI), lentiviral e.g., human immunodeficiency viruses (HIV), simian immunodeficiency virus (SIV), and feline immunodeficiency virus (FIV)), herpes simplex virus, cytomegalovirus, picornavirus (e.g. Rhinovirus, Poliovirus etc), baculovirus vectors (Autographa californica multiple nucleopolyhedrovirus (AcMNPV), hepatitis B virus (HBV), rubulavirus (new castle disease virus), parainfluenza virus, influenza virus, respiratory syncytial virus (RSV), human metapneumovirus (hMPV), respiratory Coronavirus (CoV), human SARS Coronavirus, Ebola, Marburg, Nipah, Chikungunya, Chandipura virus, Rotavirus, Human papilloma virus, Herpes simplex virus, herpes simplex virus type 1 (HSV-1), Hepatitis A, Hepatitis C, Hepatitis B, Hepatitis E, Poliovirus, Variola Virus (e.g. smallpox, Monkeypox) and Varicella virus antigens.

In one of the embodiments of the present embodiment, bacterial antigen may be processed and purified to obtain capsular polysaccharide and further conjugated to one or more carrier protein selected from group comprising but not limited to CRM197, diphtheria toxoid, tetanus toxoid, Neisseria meningitidis outer membrane complex, fragment C of tetanus toxoid, recombinant full-length tetanus toxin with eight individual amino acid mutations (8MTT), pertussis toxoid, protein D of H. influenzae, E. coli LT, E. coli ST, exotoxin A from Pseudomonas aeruginosa, outer membrane complex c (OMPC), porins, transferrin binding proteins, pneumolysin, pneumococcal surface protein A (PspA), pneumococcal surface adhesin A (PsaA), PhtA, PhtB, PhtE, pneumococcal PhtD, pneumococcal surface proteins BVH-3 and BVH-11, M. catarrhalis uspA, protective antigen (PA) of Bacillus anthracis and detoxified edema factor (EF) and lethal factor (LF) of Bacillus anthracis, ovalbumin, keyhole limpet hemocyanin (KLH), C5a peptidase group A or group B Streptococcus, human serum albumin, bovine serum albumin (BSA), purified protein derivative of tuberculin (PPD), Cholera toxin subunit B, fHbp, Por A and Por B.

In another embodiment of the present disclosure, the bacterial antigen may be Neisseria meningitidis serogroup A, C, W, Y and X, and further the polysaccharide of the respective serogroups may be conjugated to a carrier protein selected from CRM197 and/or Tetanus toxoid.

The bacterial antigens of the present disclosure may be conjugated to the carrier protein using an appropriate conjugation chemistry, including, but not limited to, cyanylation chemistry, CNBr chemistry, reductive amination chemistry, and carbodiimide chemistry. The cyanylation agent may include, but is not limited to, 1-cyano-4-dimethylaminopyridinium tetrafluroborate (CDAP), 1-cyano-4-pyrrolidinopyridinium tetrafluorborate (CPPT), 1-cyano-imidazole (1-CI), 1-cyanobenzotriazole (1-CBT), 2-cyanopyridazine-3 (2H) one (2-CPO), and a functional derivative or modification thereof.

In one embodiment of the present disclosure, the antigen is conjugated to the carrier protein in the presence of a linker. The linker may be selected from the group comprising hexanediamine, ethylene diamine, hydrazine, adipic dihydrazide, and 1,6-diaminooxyhexane.

In another embodiment of the present disclosure, the antigen is conjugated to the carrier protein in the absence of a linker.

In an embodiment, the vaccine composition may comprise excipients selected from stabilizers, pharmaceutically acceptable transporter, binder, carrier, isotonic agent, emulsifier, humectant, surfactants, polymers, preservatives, diluents, and salts.

Examples of the stabilizers may include carbohydrate, selected from the group comprising, but not limited to, natural carbohydrates, synthetic carbohydrates, polyols, glass transition facilitating agents, monosaccharides, disaccharides, trisaccharides, oligosaccharides and their corresponding sugar alcohols, polyhydroxyl compounds such as carbohydrate derivatives and chemically modified carbohydrates, hydroxyethyl starch and sugar copolymers. Both natural and synthetic carbohydrates are suitable for use. Synthetic carbohydrates include, but are not limited to, those which have the glycosidic bond replaced by a thiol or carbon bond. Both D and L forms of the carbohydrates may be used. The carbohydrate may be non-reducing or reducing. Where a reducing carbohydrate is used, the addition of inhibitors of the Maillard reaction is preferred. Reducing carbohydrates suitable for use in the composition are those known in the art and include, but are not limited to, glucose, sucrose, maltose, lactose, fructose, galactose, mannose, maltulose and lactulose. Non-reducing carbohydrates include, but are not limited to, non-reducing glycosides of polyhydroxyl compounds selected from sugar alcohols and other straight chain polyalcohols. Other useful carbohydrates include raffinose, stachyose, melezitose, dextran, cellobiose, mannobiose and sugar alcohols. The sugar alcohol glycosides are preferably monoglycosides, in particular the compounds obtained by reduction of disaccharides such as lactose, maltose, lactulose and maltulose. Glass forming agent is selected from the group consisting of sucrose, mannitol, trehalose, mannose, raffinose, lactitol, lactobionic acid, glucose, maltulose, iso-maltulose, maltose, lactose sorbitol, dextrose, fructose, glycerol, or a combination thereof.

Examples of the stabilizers may include amino acids selected from the group, but not limited to, tricine, arginine, leucine, iso-leucine, histidine, glycine, glutamine, lysine, L-alanine, peptide, hydrolysed protein or protein such as serum albumin.

Examples of the stabilizers may include hydrolyzed protein selected from a group consisting of gelatin, lactalbumin hydrolysate, monosodium glutamate, collagen hydrolysate, keratin hydrolysate, peptides, Casein hydrolysate and whey protein hydrolysate or protein such as serum albumin.

As used herein, the term “gelatin” means a sterile non-pyrogenic protein preparation (e.g., fractions) produced by partial acid hydrolysis (type A gelatin) or by partial alkaline hydrolysis (type B gelatin) of animal collagen, most commonly derived from cattle, pig, and fish sources. Gelatin can be obtained in varying molecular weight ranges. Recombinant sources of gelatin may also be used.

Examples of surfactants may include non-ionic surfactants such as polysorbate 20, polysorbate 40, polysorbate 60, polysorbate 65, polysorbate 80, polysorbate 85, nonylphenoxypolyethoxethanol, octylphenoxypolyethoxethanol, oxtoxynol 40, nonoxynol-9, triethanolamine, triethanolamine polypeptide oleate, polyoxyethylene-660 hydroxystearate, polyoxyethylene-35 ricinoleate, soy lecithin and a poloxamer.

Examples of polymers may include but is not limited to, dextran, carboxymethylcellulose, hyaluronic acid and cyclodextrin. Examples of the salts may include NaCl, KCl, KH₂PO₄, Na₂HPO₄·2H₂O, CaCl₂, MgCl₂ and the like.

Examples of preservatives include, but is not limited to, 2-phenoxyethanol, benzethonium chloride (Phemerol), phenol, m-cresol, thiomersal, formaldehyde, methyl paraben, propyl paraben, benzalkonium chloride, benzyl alcohol, chlorobutanol, p-chlor-m-cresol, benzyl alcohol, and combinations thereof.

In an embodiment of the present disclosure, r-Tbp-B may be used as an adjuvant in vaccine compositions. A vaccine composition comprising an antigen and r-Tbp-B as an adjuvant aids in the slow release of the target antigen. When r-Tbp-B as an adjuvant is injected with the antigen of interest, r-Tbp-B has been shown to induce significant B and T cell responses. One of the features of r-Tbp-B is that it slowly releases the antigen when injected in a subject, allowing the antigen to be exposed to the immune system for a longer period of time, resulting in a higher T and B cell response.

In another embodiment of the present disclosure, r-Tbp-B may be used as a carrier protein. Recombinant Tbp-B an alternative carrier protein in vaccine compositions comprising glycoconjugates may overcome bystander interference which masks the immunological epitopes of polysaccharides due to repeated use of conventional carrier protein (such as tetanus toxoid (TT), diphtheria toxoid (DT), Cross Reacting Material 197 (CRM197), etc).

In an embodiment of the present disclosure, the vaccine composition comprises multiple distinct pneumococcal polysaccharides conjugated to r-Tbp-B as carrier protein; 125 μg/dose Al³⁺ (aluminium phosphate gel); histidine (5 mM to 30 mM); succinic acid (10 mM to 30 mM), sodium chloride (0.9% to 3%), polysorbate-20 (1% to 5%), thiomersal (1% to 5%); and pH 5.8.

The method of the present disclosure uses a single chromatography step for purification of r-Tbp-B after cytoplasmic expression in Escherichia coli, compared to known methods wherein multiple chromatographic methods are used which is tedious, labor intensive and time consuming. The vaccine composition containing such r-Tbp-B elicits at least 2-fold higher IgG titers when recombinant transferrin binding protein is present in the composition.

### EXAMPLES

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following examples which are set forth for illustration purpose only and not to be construed for limiting the scope of the disclosure.

### Source of Biological Resources Used in the Present Disclosure:

All the Biological Materials Used in the Invention are Obtained from Outside India.
Transferrin binding protein sequence from Neisseria meningitidis C (FAM18) was sourced from UniProt ID-A1KVG7 (Organism: Neisseria meningitidis serogroup C/serotype 2a (strain ATCC 700532/DSM 15464/FAM18). UniProt is the Universal Protein resource combining European Molecular Biology Laboratory-European Bioinformatics Institute (EMBL-EBI), UK; Swiss Institute of Bioinformatics (SIB), Switzerland; Protein Information Resource (PIR), US databases.
Vector: pET 30a (+) was procured from Gene Universal Inc., Newark Delaware, USA.
Host System: E. coli BL-21/DE3 competent cells (BL21 (DE3) Competent Cells. Cat. number: EC0114) for plasmid transformation were purchased from Thermo Scientific™, California, USA.
Transferrin binding protein standard was procured from MyBioSource, Inc., San Diego, California, USA.
Pneumococcal polysaccharides: Streptococcus pneumoniae polysaccharides from serotypes 3, 6A, 19A and 19F were procured from American Type Culture Collection (ATCC), Manassas, Virginia, near Washington DC, USA or from Centers for Disease Control and Prevention (CDC), Atlanta, USA.
Tetanus Toxoid (TT): The strain Clostridium tetani Harvard Strain No. 49205 was obtained from The Rijks Institute Voor de Volksgezondheid (Netherlands) by the National Control Authority C.R.I. Kasauli, in Lyophilized form.
CRM197: CRM197 is obtained from Recombinant Strain CS463-003 (MB101) of Pseudomonas fluorescens procured from Pfenex USA.
Peroxidase-conjugated ChromPure Human Transferrin was procured from Jackson Immunoresearch Laboratories Inc., Pennsylvania, USA.
Baby Rabbit complement (3-4 week sterile) is procured from Pel Freez Biologicals, 205 North Arkansas Street Rogers, AR 72756, US. {Cat.No. 31061-3}.

Luria Broth (HiMedia) was used as per the manufacturer's recommendation for the fermentation of E. coli.

### Example-1: Optimization Studies

Optimization of different parameters, such as sonication condition and IPTG (inducing agent) concentration was carried out.

### Optimization of Sonication Parameter:

Sonication was carried at different amplitudes (25, 50, and 70) and time periods (3 minutes, 5 minutes) to determine the optimum sonication condition.

TABLE 1
Amplitude optimization

| Time | Protein (mg/ml) |
|---|---|
| 25 Amplitude- 5 minutes | 1.26 |
| 70 Amplitude- 5 minutes | 2.23 |
| 70 Amplitude- 3 minutes | 1.56 |
| 50 Amplitude- 3 minutes | 1.34 |

All experiments were performed in cold condition

Observation: It is seen from Table-1 that sonication at 70 amplitude-5 minutes was found to be optimal, where complete lysis of the cell was achieved and maximum protein expression was observed.

### Inducing Agent (IPTG) Optimization:

Optimization for an inducing agent: Isopropyl ß-D-1-thiogalactopyranoside (IPTG), was carried out. 1 mM, 0.5 mM, and 0.1 mM IPTG concentrations were studied, and 1 mM IPTG was found to be optimal for cell growth and expression of a recombinant protein which was eventually used for further experiments.

TABLE 2
IPTG Optimization

| IPTG | OD at 590 nm | |
|---|---|---|
| Concentration | (After 4 hours) | Protein Expression |
| 0.1 mM | 2.29 | * |
| 0.5 mM | 1.96 | ** |
| 1.0 mM | 2.12 | ***** |
| Control | 2.85 | No expression |

*Grade for expression

Observation: It is seen from Table-2 that although cell mass with 0.1 mM was higher than 1.0 mM IPTG concentration, an elevated expression of recombinant protein was observed for 1.0 mM IPTG concentration.

### Example-2: Cloning and Transformation of the Transferrin Binding Protein Gene in Host System

After optimization of sonication condition and IPTG concentration, recombinant transferrin binding protein was cloned and expressed in a suitable host system.

The Tbp-B sequence from Neisseria meningitidis C (FAM18) was sourced from UniProt ID-A1KVG7 (Organism: Neisseria meningitidis serogroup C/serotype 2a (strain ATCC 700532/DSM 15464/FAM18).
Glycerol stock of Cytoplasmic construct {host system comprising the cloning vector with the gene sequence encoding r-Tbp-B} was grown overnight in Luria broth (LB) with 30 μg/ml kanamycin at 37° C./220 RPM (Inoculum development);
Cells were allowed to grow at 37° C., 220 rpm till OD reached 0.5-0.7;
Cells were Induced with IPTG final concentration of 1 mM;
Allowed to grow at 37° C./220 rpm for 4 hours;
Culture was centrifuged at 10000 rpm for 10 minutes at 4° C. to collect pellet which contains inclusion bodies containing transferrin binding protein.

### Example-3: Purification of Transferring Binding Protein

The cloned and transformed recombinant transferrin binding protein was harvested and further subjected to downstream processing. The process steps for Tbp-B extraction/harvesting and purification is provided below:

### IB Washing Protocol

200 mg pellet and added 40 ml lysis buffer
(Lysis buffer-50 mM Tris-C1, 50 mM EDTA, 15% sucrose, pH 8)
Added lysozyme 5 mg/ml, mixed well, and incubated for 1 hr at 37° C., 180 RPM
Sonicated at 100 Amp for 5 min (5 Second ON, 5 Second OFF)
Culture was centrifuged at 10000 rpm for 10 min at 4° C.
Decanted supernatant
To the pellet, added 40 ml wash buffer (0.5 M NaCl, 2% Triton X-100)
Mixed well and culture was spun at 10000 rpm for 10 min at 4° C.
Decanted supernatant
In pellet, added 40 ml 0.5 M NaCl
Mixed well and spun culture, 10000 rpm for 10 min at 4° C.
Decanted supernatant
In pellet added 40 ml of Distilled water
Mixed well and spun culture, 10000 rpm for 10 min at 4° C.
Decanted supernatant
Mixed well and spun culture, 10000 rpm for 10 min at 4° C.
Finally, IB is solubilized in Tris-Cl buffer pH 8 (50 mM Tris, 500 mM NaCl, pH 8) at 37° C. for 2 hrs
Spun culture, 10000 rpm for 15 min at 4° C.
Separated supernatant (Soluble) and pellet (Insoluble-IB)
SDS PAGE of solubilized and insolubilized fractions

The cytoplasmic construct is the molecularly engineered gene sequence clone that is expressed in E. coli with no signal sequence. The cytoplasmic construct will express desired cloned protein which will directly settle in the cytoplasm as it does not have any signal for any further movement like periplasm etc.

The recombinant Tbp-protein of the present disclosure was expressed in Inclusion Body (IB) and solubilization of IB was carried out before chromatographic purification of Tbp-B proteins as per the process steps provided below:

### IB's Solubilization

Pellet (In-soluble-IB) dissolved in 6 ml sonication buffer.
   (Sonication buffer—100 mM Tris-C1, 50 mM Glycine pH 8.0)
Sonicated at 100 Amp for 1 min (5 Second ON, 5 Second OFF)
To the solution added 20 ml dispersion buffer
   (Dispersion buffer—100 mM Tris-Cl, 50 mM Glycine, 8M Urea pH 8.0)

A major part of the Tbp was an inclusion body hence solubility was achieved by the addition of 8 M Urea which was subsequently diluted further and finally completely removed by 10 kDa cut-off dialysis cassettes. Cut-off membrane sufficiently removes urea which has molar mass of 60.0 g/mol, before further processing.

### Anion Exchange Chromatography (AEX)

IB's in dispersion buffer was dialyzed overnight (at 4° C.) with 3 exchanges of buffer (50 mM Tris, 100 mM NaCl, pH 8) to allow protein refolding.
10 ml of Dialyzed sample was loaded on Macro-Prep® High Q Media (Bio-Rad) anion exchange resin packed in syringe column (6 ml bed volume). Before that column was equilibrated with an equilibration buffer for around 10 column volume (CV) (Equilibration buffer—50 mM Tris-Cl pH 8.6).
Flow-through sample (unreacted) was collected and a further 7 CV wash was done with column wash buffer.
   (Wash buffer-50 mM Tris-Cl pH 8.6)
Elution was done with elution buffer-1 and elution buffer-2 and elutes were collected at different elution times.
   (Elution buffer-1—50 mM Tris-C1, 200 mM NaCl pH 8.6)
   (Elution buffer-2—50 mM Tris-C1, 500 mM NaCl pH 8.6)

Known methods for purification of Tbp used affinity chromatography, in which crude Tbp was allowed to interact with CNBr activated sepharose 4B coupled to Human transferrin (HTf) and eluted gradient-wise, which was further followed by ion-exchange chromatography or Size exclusion chromatography. The purification of Tbp in accordance with the present disclosure involves a thorough IB wash followed by ion-exchange chromatography (Anion exchange) with a purity of >95%. Table-3 provides the comparison of purification of Tbp by previously known methods and the method of the present disclosure.

TABLE 3
Comparison of previously known purification and method of present disclosure

| Purification of Tbp in | | Advantages of purification |
|---|---|---|
| accordance with | Purification of Tbp in | of Tbp in accordance with |
| previously known | accordance with the | the method of present |
| methods | present disclosure | disclosure |
| Used three | Thorough washing was | rTbp-B was not tagged, |
| chromatography steps | applied to Inclusion bodies | hence tag cleavage was |
| while purifying | (IB's) which removes most of | not required. |
| recombinant Tbp-B | the contaminants present in | Fewer steps of |
| which includes affinity | the mixture. | chromatography were |
| chromatography followed | Cleaned IB's was dialyzed | involved. |
| by ion exchange and | overnight using 10 kDa cut- | Time required for the |
| finally size exclusion | off dialysis membrane which | entire process was reduced |
| chromatography. | further cleaned up Tbp from | and easy to handle. |
| Also, these methods used | impurities. | Though the number of |
| the tagged method for | Semi-purified IB's was | chromatography steps was |
| protein purification. | applied on the anion exchange | reduced, the purity of r- |
| (e.g. His-tag, MBP-tag, | column (single | Tbp-B obtained was >95%. |
| etc.) | chromatography step) which | |
| | was eventually eluted with a | |
| | gradient of salt resulting | |
| | in ~65 kDa purified Tbp. | |

Known methods for Tbp-B used multiple chromatography steps wherein resin such as cyanogen bromide (CNBr) sepharose was coupled to the Tbp-B, followed by cation chromatography and further using gel filtration chromatography (Table-3). Another drawback of using CNBr activated resin is the leaching observed during chromatographic elution which interferes with biochemical and immunological analysis.

On the other hand, the method of the present disclosure used single-step purification using anion exchange chromatography achieving >95% purity of Tbp-B. It is known that use of multiple chromatography steps may increase purity of the molecule being purified. However, an approximate loss of about 20 to 30% of the molecule is observed in each stage of chromatography. The method of the present disclosure using a single step chromatography provide Tbp-B with desirable purity without significant loss. Further, the present method avoids the drawbacks associated with the use of CNBr activated resins. FIG. 1 compares the fractions obtained with previously known CNBr activated resins and anion exchange chromatography method of the present disclosure, wherein Lane-M is Molecular weight Standard (180 kDa to 10 kDa, higher to lower molecular weight); Lane-1 is Crude Protein; Lane-2 is fraction diafiltered by 10 kDa cut-off Membrane; Lane-4 is Anion exchange Elute-1 fraction; Lane-5 is CNBr activated resin Elute fraction; and Lane-6 is Anion exchange Elute-2 fraction.

It is seen from FIG. 1 that the Lane-5 elute collected from CNBr activated sepharose resin coupled with Tbp-B contains low molecular weight impurities which were not removed even after size exclusion resin whereas Lane-4 and Lane-6 fractions obtained using Anion exchange Macro-prep resin as per the method of the present disclosure yielded pure fraction of Tbp-B with no impurities even using a single-step purification process.

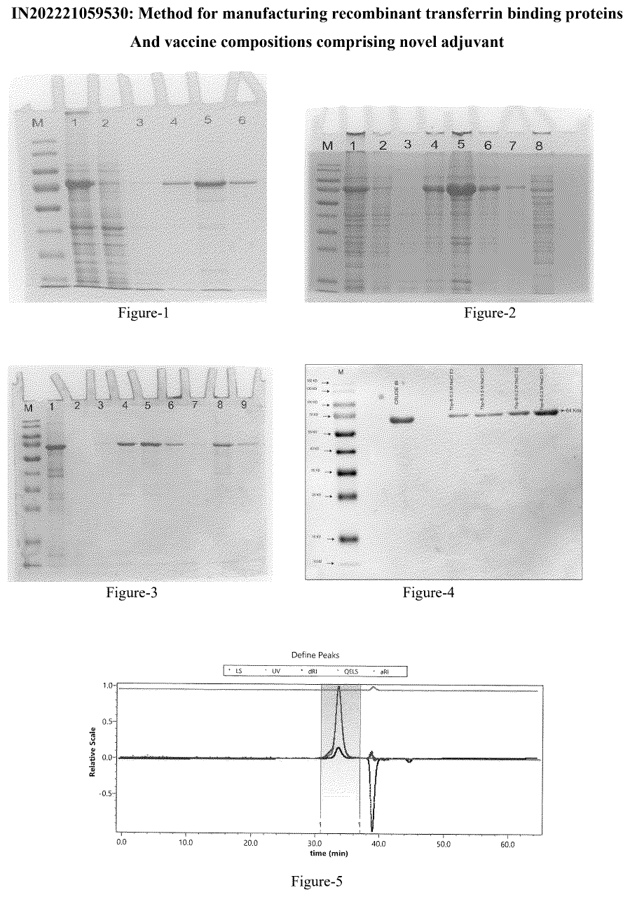

FIG. 2 shows the fractions obtained using tagged protein and the fractions obtained using the method of the present disclosure without tagging Tbp-B: M is Molecular weight standard 180 kDa to 10 kDa, higher to lower molecular weight; Lane-4 and Lane-6 are His-tagged elutes of Tbp-B; Lane-7 is non-tagged Macro prep Anion exchange elute (in accordance with the present disclosure); Lane-5 is crude protein before loading on to the column.

It is clearly seen from FIG. 2 that the purity of the fraction in Lane-7 (which is non-tagged r-Tbp-B) is significantly better that the fractions in the other Lanes.

Tagging of recombinant proteins are routinely used for protein research and many different types of affinity tags are available, such as bigger fusion-protein tags like glutathione S-transferase (GST), maltose binding protein (MBP), and green fluorescent protein (GFP). A poly-histidine tag (His-tag) or a strep tag, among others, may instead be added to the recombinant target protein. Depending on the target protein's characteristics (such as the existence of downstream cleavage sites, post-translational modification sites, or subcellular localization sequences), these tags are frequently appended to either the N- or C-terminus. However, addition of a non-native peptide or fusion protein tag may result in negative effects.

### Some of the Major Disadvantages of the Tagged Protein (Utilized by Previously Reported Purification Methods) Include:

The tag may interfere with the structure and biological activity of the protein. The structural and functional properties of the target protein may be altered by the insertion of protein tags, particularly bigger tags. The addition of a tag may result in a partial or total loss of functionality.
The tag may need to be removed depending on the downstream application resulting in instability of the protein after tag removal.
Larger tags impose a heavy metabolic burden on the expression host.
The effective removal of the tags from recombinant proteins effectively can be difficult and time-consuming. Usually, a protease is used, and a protease cleavage site is placed between the tag and protein. The cleavage sequence may further have an effect on the target protein's structure and/or function.

The method of the present disclosure did not tag the recombinant transferrin binding protein and further used a single step anion exchange chromatography to obtain a purity of >95%.

### Example-4: Characterization of Purified Transferrin Binding Protein

The physico-chemical characteristics of elutes were carried out using different methods:

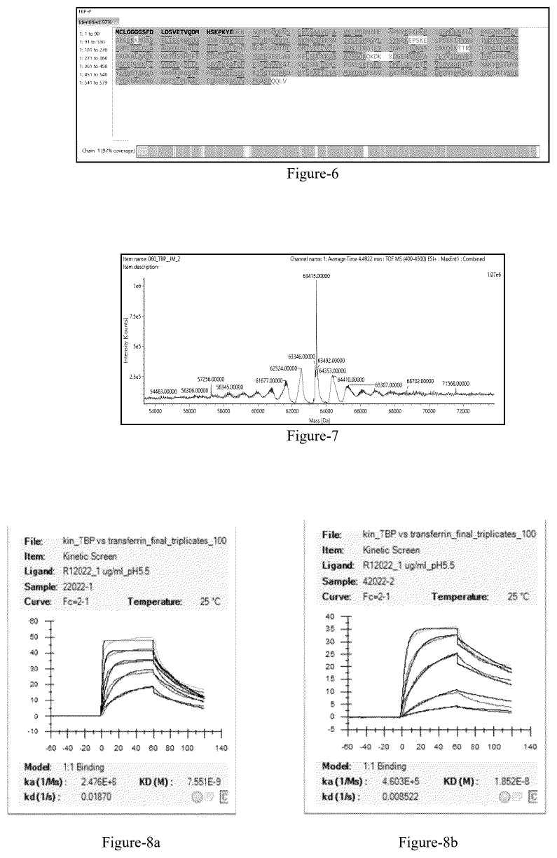

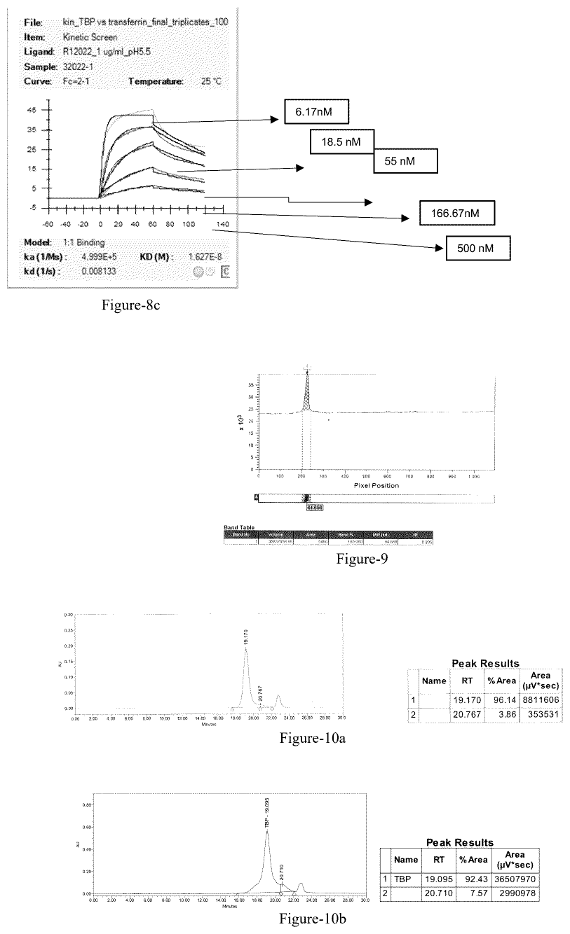

Purified recombinant Tbp-B (r-Tbp-B) was analyzed by SDS-PAGE, SEC-MALS, and confirmed by Western blot when allowed to react with Transferrin-HRP antibody. Protein samples were analyzed for N-terminal sequencing and intact mass from National Chemical Laboratory (NCL), Pune, Maharashtra, India.
A) SDS-PAGE: The SDS PAGE for Anion Exchange chromatography elutes collected at different salt concentration gradients is illustrated in FIG. 3: Lane-M is Molecular weight standard, higher to lower 180 kDa to 10 kDa, Lane-1 is Crude Tbp-B, Lanes 4-6 are purified fraction of Tbp-B protein having molecular weight of ~63 to 65 kDa.
B) Western Blotting: It is seen that few low molecular bands are from Host cell protein (HCP) of E. coli which contributes <5% of impurities. FIG. 4 shows the Western Blotting analysis: M is molecular weight standard, higher to lower 180 kDa to 10 kDa, Crude Tbp-B, different purified fraction of Tbp-B protein of ~65 kDa. It is seen from FIG. 4 that the Tbp-B showed strong binding to transferrin—HRP in all the elutes, resulting in specific binding to all elutes of pure protein and the absence of non-specific bands.
C) Size exclusion chromatography with Multi-angle laser light scattering (SEC-MALS) is shown in FIG. 5, which reveals an absolute molecular mass of 64 kDa. SEC-MALS gave cumulative response from all the four detectors i.e. LS (light scattering-A), QELS (Quazi light scattering-A), UV (ultra-violet-C), dRI (differential refractive index-B).
D) Peptide fingerprinting was carried out to determine the sequence homology and is illustrated in FIG. 6. It is seen from FIG. 6 that 97% sequence homology was observed in expressed protein against the cloned sequence by peptide fingerprinting using LC-MS-MS.
E) Mass Spectrometry: The intact mass of r-Tbp-B was determined by Mass Spectrometry using LC-MC-MS as shown in FIG. 7. It is seen from FIG. 7 that the intact mass of the Tbp-B was found to be 63.34 kDa.
F) Surface Plasmon resonance (SPR): Protein integrity of the Tbp-B obtained by the method of the present disclosure was analyzed by SPR (SPR was carried out Bia-core SPR technology) as shown in FIGS. 8a-8c.
   The degree of a single molecule's binding to its ligand is measured by its affinity. The equilibrium dissociation constant (KD), which was used to assess and rank the order strengths of bimolecular interactions, is employed to quantify and report in SPR. Various Tbp-B samples (0.2M NaCl elute, 0.5M NaCl elute, and partially purified) were analyzed by Biacore T200 to evaluate kinetic parameters for Holo-transferrin binding to transferrin binding protein, ON-rates (Kₒₙ)/OFF-rates (K_{off}), and the equilibrium kinetic constant of dissociation (KD). The results obtained are illustrated in FIGS. 8a-8c and Table-4.

TABLE 4
Surface Plasmon resonance

| | ka (1/Ms) | | | | | |
|---|---|---|---|---|---|---|
| Sample Name | Run-1 | Run-2 | Run-3 | Average | STDEV | % CV |
| 0.2M Elute NaCl | 2.5E+06 | 2E+06 | 2E+06 | 2E+06 | 55076 | 2.27 |
| 0.5M Elute NaCl | 5.0E+05 | 4E+05 | 5E+05 | 5E+05 | 32868 | 6.99 |
| Partially purified | 4.5E+05 | 5E+05 | 4E+05 | 4E+05 | 26633 | 6.02 |
| | kd (1/s) | | | | | |
| Sample Name | Run-1 | Run-2 | Run-3 | Average | STDEV | % CV |
| 0.2M Elute NaCl | 0.0187 | 0.019 | 0.02 | 0.019 | 5E−04 | 2.6 |
| 0.5M Elute NaCl | 0.0081 | 0.009 | 0.009 | 0.009 | 5E−04 | 5.82 |
| Partially purified | 0.0083 | 0.009 | 0.009 | 0.009 | 3E−04 | 3.77 |

| | KD (M) | | | | | |
|---|---|---|---|---|---|---|
| Sample Name | Run-1 | Run-2 | Run-3 | Average | STDEV | % CV |
| 0.2M Elute NaCl | 7.55E−09 | 8.1E−09 | 8.1E−09 | 7.92E−09 | 3.205E−10 | 4.0 |
| 0.5M Elute NaCl | 1.63E−08 | 2.1E−08 | 1.9E−08 | 1.86E−08 | 2.255E−09 | 12.1 |
| Partially purified | 1.83E−08 | 1.9E−08 | 2.2E−08 | 1.95E−08 | 1.966E−09 | 10.1 |

Five distinct dilution concentration standards of Tbp-B, including 6.17 nM, 18.5 nM, 55 nM, 166.67 nM, and 500 nM, were used to run all of the tests in triplicate

For the samples of 0.2M NaCl elute, 0.5M NaCl elute, and partially purified sample, we carried out both multi-cycle experiments and calculated the Equilibrium kinetics constant of dissociation (KD), for which the values were found to be 7.92 nM, 18.6 nM, and 19.5 nM correspondingly. Five distinct dilution concentration standards of Tbp-B, including 6.17 nM, 18.5 nM, 55 nM, 166.67 nM, and 500 nM, were used to run all of the tests in triplicate. Table-4 illustrates the equilibrium kinetics constant of dissociation (KD), association constant (Ka), and dissociation constant (Kd) for all the samples analyzed. It is evident that the 0.2M NaCl elute sample has a very low KD value, indicating a strong association with the ligand-protein transferrin. Although the KD values for the other two samples demonstrate favorable results, they demonstrate a lower affinity for receptor protein. % CV for all the samples analyzed was well within the acceptance criteria i.e. less than 15%.
G) Densitometry Analysis: FIG. 9 illustrates the densitometry analysis of r-Tbp-B. With a molecular weight of 65 kDa, densitometry analysis revealed 100 percent band purity.

The physico-chemical analysis of the Tbp-B obtained by the method of the present disclosure is summarized in Table-5.

TABLE 5
Physico-chemical analysis of the Tbp-B

| S. No | Method of Analysis | Remarks |
|---|---|---|
| 1 | SDS-PAGE | A molecular weight of around 63 kDa was |
| | | observed |
| 2 | Western Blotting | Transferrin-HRP antibodies bind specifically to |
| | | transferrin-binding protein bands. |
| 3 | Size exclusion chromatography with | The absolute molecular weight of r-Tbp-B was |
| | Multi-angle laser light scattering (SEC- | found to be 64 KDa |
| | MALS) | |
| 4 | Peptide fingerprinting | 97% sequence homology was observed in |
| | | expressed protein against the cloned sequence. |
| 5 | Intact mass analysis by LC-MS | The intact mass of r-Tbp-B was found to be |
| | | 63.34 KDa |
| 6 | Surface Plasmon resonance (SPR) | Protein integrity was confirmed using Bia-core |
| | (carried out by Bia-core, Sweden) | SPR technology. |

### Example-5: Formulations Comprising Purified Transferrin Binding Protein

Pneumococcal conjugates were formulated using purified transferrin binding protein obtained by the method of the present disclosure.

The formulations studied include r-Tbp-B as carrier protein/adjuvant/antigen (immunogen) along with different pharmaceutically acceptable excipients.

The details of the formulations are provided in the following sections. In one instance the vaccine composition comprises multiple distinct pneumococcal polysaccharides conjugated to r-Tbp-B as carrier protein; 125 μg/dose Al³⁺ (aluminium phosphate gel); histidine (5 mM to 30 mM); succinic acid (10 mM to 30 mM), sodium chloride (0.9% to 3%), polysorbate-20 (1% to 5%), thiomersal (1% to 5%); and pH 5.8.

### Example-6: Stability Studies of Purified Transferrin Binding Protein

### Transferrin Binding Protein as Adjuvant:

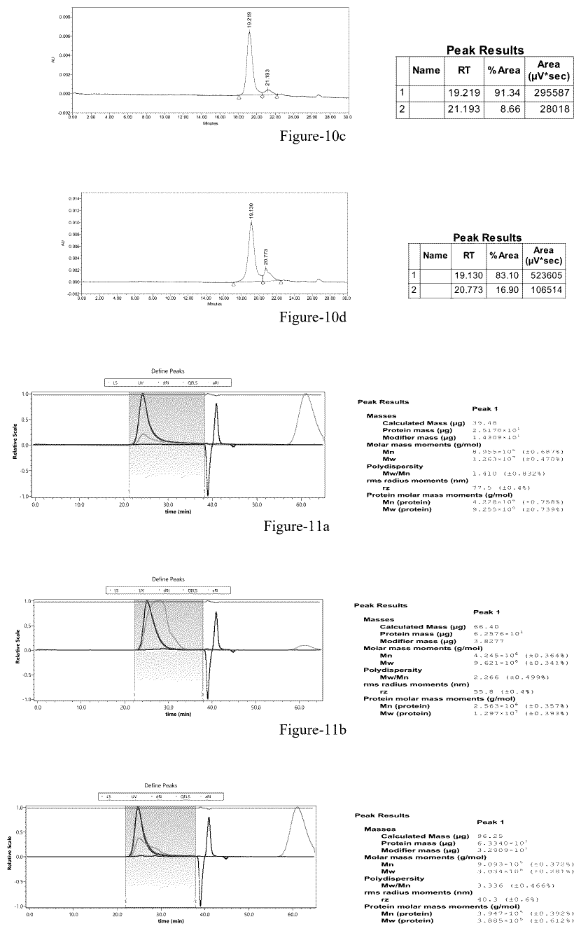

Stability studies for the purified r-Tbp-B was carried out at 2-8° C. for 1 month, 3 months and 6 months; and at 25° C. for 1 month. The results obtained are illustrated in FIGS. 10a-10d and Table-6.

TABLE 6
Stability studies at 2-8° C.

| S. No | Stability Condition | % Purity by SEC-HPLC |
|---|---|---|
| 1 | Tbp-B 1 Month 2-8° C. | 96.14 |
| 2 | Tbp-B 3 Month 2-8° C. | 92.43 |
| 3 | Tbp-B 6 Month 2-8° C. | 91.34 |
| 4 | Tbp-B 1 Month 25° C. (Accelerated) | 83.10 |

FIGS. 10a-10d and Table-6 illustrates the percent purity data of Tbp-B by SEC-HPLC. Tbp-B obtained by the method of the present disclosure was found to be very stable (with at least 90% purity retained) at 2-8° C. up to 6 months and a slight decrease in purity was recorded at accelerated temperature conditions.

### Transferrin Binding Protein as Carrier Protein:

Streptococcus pneumoniae polysaccharides (6A, 19A, 22F) conjugated with r-Tbp-B were tested for polysaccharide (Ps), free Polysaccharide content, molecular size (SEC-HPLC), and absolute molecular weight using multi-angle laser light scattering. The stability data of these conjugates were performed at 2-8° C. and 25° C. for 1 month, 4 months, and 3 months, respectively.

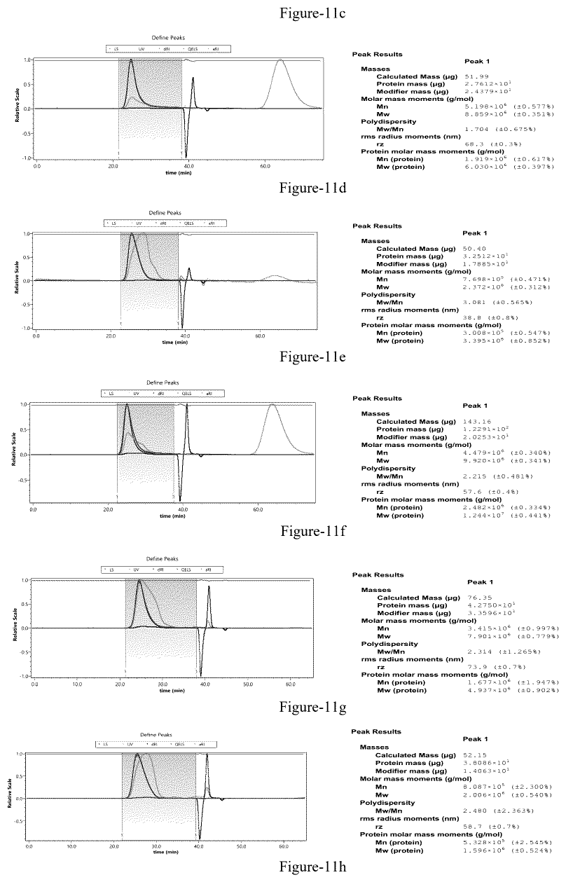

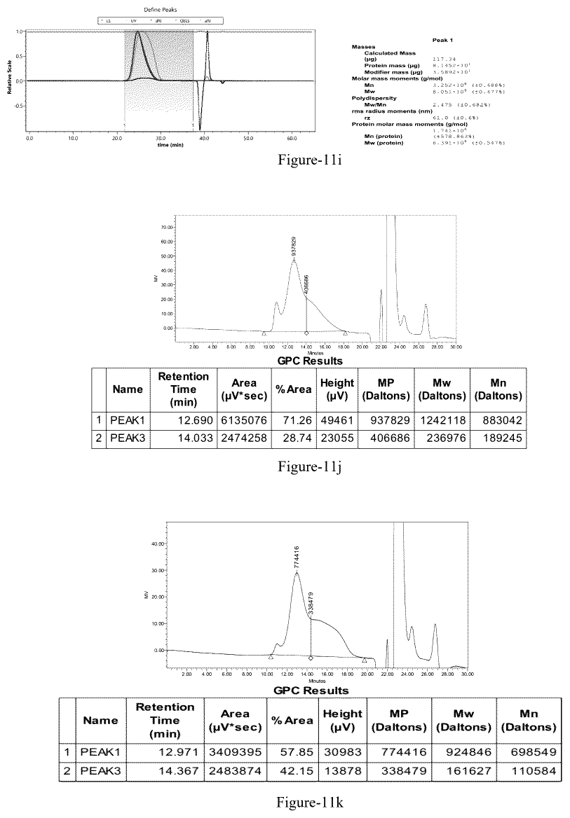

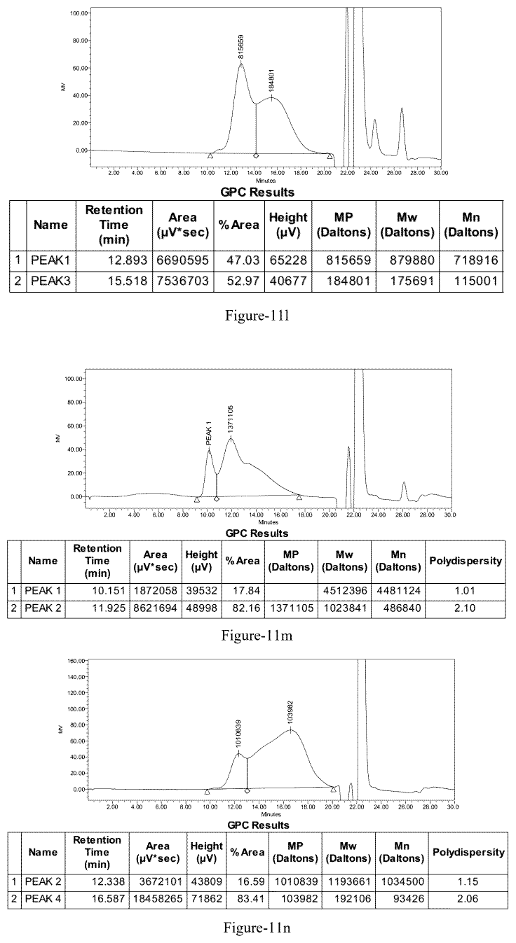

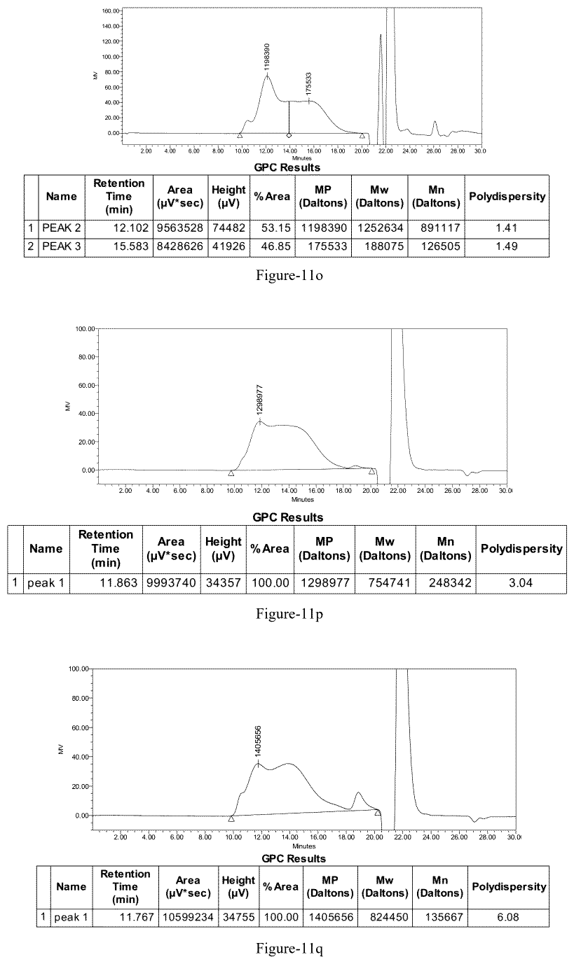

The formulation detail and the result obtained are provided in Table-7 and FIGS. 11a-11r illustrate the stability analysis result for Tbp-B as carrier protein.

TABLE 7
Stability studies at 2-8° C.

| | | | | MW | Absolute |
|---|---|---|---|---|---|
| | Ps | Free Ps | Ps:Pr | (SEC- | MW |
| Study Parameter | (mg/ml) | (%) | Ratio | HPLC) | (MALS) |
| 2-8° C. (1 Month) | | | | | |
| Pnu-type 6A-Tbp-B CJ | 1.58 | 7.71 | 1.58 | 1242 | 12263 |
| Pnu-type 19A-Tbp-B CJ | 1.97 | 17.05 | 0.80 | 924 | 9621 |
| Pnu-type 22F-Tbp-B CJ | 1.54 | 23.92 | 1.44 | 879 | 3034 |
| 2-8° C. (4 Months) | | | | | |
| Pnu-type 6A-Tbp-B CJ | 1.58 | 9.23 | 1.58 | 1023 | 8859 |
| Pnu-type 19A-Tbp-B CJ | 1.97 | 19.24 | 0.80 | 1193 | 2372 |
| Pnu-type 22F-Tbp-B CJ | 1.54 | 39.29 | 1.44 | 1252 | 9920 |
| 25° C. (3 Months) | | | | | |
| Pnu-type 6A-Tbp-B CJ | 1.58 | 10.90 | 1.58 | 754 | 7901 |
| Pnu-type 19A-Tbp-B CJ | 1.97 | 22.52 | 0.80 | 824 | 2006 |
| Pnu-type 22F-Tbp-B CJ | 1.54 | 42.23 | 1.44 | 956 | 8051 |

Pnu = pneumococcal polysaccharide;
CJ = pneumococcal polysaccharide-carrier protein conjugate;
MW = Molecular weight

Table-7 includes the data of pneumococcal polysaccharide serotypes 6A, 19A, and 22F conjugated to Tbp-B/CRM/TT that were analyzed for polysaccharide, free polysaccharide (Ps), polysaccharide to carrier protein (Ps:Pr) ratio, molecular size (SEC-HPLC and MALS). The studies were conducted at 2-8° C. for 1 month and 4 months at; and at 25° C. for 3 months. Conjugates were found to be stable at 2-8° C. for 4 months whereas a slight increase in free Ps was noted at 25° C. at 3-month time point.

As seen from FIGS. 11a-11r, similar results were observed for molecular weight (SEC-HPLC) at 2-8° C. for 1 month and 4 months. An increase in size was noted at the 4-month time point indicating Ps-Ps/protein-protein aggregation resulting in the increase in molecular weight of the conjugates. Absolute molecular weight (MALS) showed a decrease at 25° C. at 3 months, whereas the absolute molecular weight (MALS) was found to be stable at 2-8° C. at 4 months.

Thus, the data illustrates the overall stability of transferrin binding protein in formulations when used as a carrier protein.

### Example-7: Immunogenicity Studies of Formulations Comprising Purified Transferrin Binding Protein as Adjuvant/Carrier Protein

Multiplexed bead-based assay (MBBA) was used to evaluate IgG titers and opsonophagocytosis assay (OPA) was performed to determine functional antibodies in various formulations.

### Protocol for Multi-Plex Bead-Based Assay (MBBA)

In this study, various bead sets were coupled with different analytes (pneumococcal polysaccharides, CRM197, Tetanus Toxoid, Diphtheria Toxoid, etc) by different coupling methods, which were developed in-house. The results are represented by the Bio-Plex Manager 4.1.1 software in terms of median fluorescence intensity (MFI).

A 96-well multi-screen HTS filter plate (Millipore, catalogue #MSBVN1250) was pre-wet with 50 μl Luminex assay buffer (1×PBS+0.2% BSA, 0.1% Tween-20+0.05% NaN₃, pH 7.2±0.2) and was kept at room temperature for 2-5 minutes. Reference serum (if any) and test (mouse) serum dilutions were made in a 96-well round bottom dilution plate from 1:100 (Corning, catalogue #3799). Pre-immunization sera were tested in six dilutions whereas the post-immunization sera tested in 12 dilutions.

Luminex carboxylated beads conjugated with the analyte of interest (as per formulation injected into animals) were diluted in Luminex assay buffer to obtain ~3000 beads/50 μl. The buffer from the pre-wet wells of the filter plate was aspirated on a vacuum aspirator. 50 μl of diluted beads were added in each required well of the pre-wet filter plate. The plate was aspirated on a vacuum aspirator. 50 μl of each dilution of all sera samples was transferred in the filter plate wells and 50 μl diluent was added in the wells named ‘Blank’. The filter plate was incubated at 37° C. for 60 minutes (+5 minutes) with 150 RPM shaking in the dark. After incubation, the plate was aspirated and washed thrice with 100 μl Luminex assay buffer/well.

The species-specific anti-IgG antibodies conjugated with phycoerythrin (PE) (Jackson ImmunoResearch) were diluted in Luminex assay buffer. 50 μl of diluted PE conjugate was added to each well of the filter plate and the plate was incubated for 30 minutes (+5 minutes) with 150 RPM shaking in the dark. After incubation, the plate was aspirated and washed thrice with 100 μl Luminex assay buffer/well. After the final wash, 100 μl of Luminex buffer was added in each well of the filter plate and the plate was read on the Luminex system (Bio-Plex 200).

### Protocol for Multiple Opsonophagocytosis Assay (OPA/MOPA)

10 μL of diluted or undiluted test serum (heat inactivated at 56° C. for 30 minutes) was used for OPA/MOPA. Sera was serially diluted 2-fold. Appropriate bacterial dilution was prepared and 20 μl of bacterial suspension was added to each well (1000 cfu/well). Plates were incubated at 37° C. for 20-25 minutes, 5% CO₂. 40 μl of differentiated HL-60 cells (4×105 cells/well) were added to each well. 10 μl of complement source (Baby Rabbit Serum) was used. Plates were incubated at 37° C. for 45 minutes with horizontal shaking (220 RPM). Spotting of 5 μl was done onto THYE (Todd Hewitt Yeast Extract agar) agar plates. Plates were further incubated overnight at 37° C., 5% CO₂. Colony counts were performed next day.

Titer calculation: Titer was considered the reciprocal of the highest dilution showing a 50% reduction from the number of colonies in the complement control well.

rTbp-B—Transferrin Binding Protein as Adjuvant:

The details of the various formulations studied and immunogenicity result obtained is provided in Table-8a and Table-8b.

TABLE 8a
Formulations for Immunogenicity Studies

| S. No | Formulation Plan |
|---|---|
| 1 | rTbp-B alone (10 μg/dose) + Pnu Type-3 CRM conjugate |
| | (2 μg/dose) |
| 2 | rTbp-B alone (10 μg/dose) + Pnu Type-19A CRM conjugate |
| | (2 μg/dose) |
| 3 | Pnu Type-3 CRM conjugate (2 μg/dose) + Alum |
| 4 | Pnu Type-19A CRM conjugate (2 μg/dose) + Alum |
| 5 | Pnu Type-3 CRM conjugate (2 μg/dose) |
| 6 | Pnu Type-19A CRM conjugate (2 μg/dose) |
| 7 | Pnu Type-3 CRM conjugate (2 μg/dose) + fHbp (10 μg/dose |
| 8 | Pnu Type-19A CRM conjugate (2 μg/dose) + fHbp (10 μg/dose) |
| 9 | rTbp-B alone |
| 10 | fHbp alone |
| 11 | Alum alone |

Pnu = pneumococcal polysaccharide;
CJ = pneumococcal polysaccharide-carrier protein conjugate;
fHbp: factor H binding protein

TABLE 8b
Immunogenicity Studies

| | | | | *MBBA | |
|---|---|---|---|---|---|
| | | | | (IgG titers- | OPA |
| Formulation Details | Code | Bleed Day | | GMT) | Value |
| Pnu Type-3-CRM CJ with rTbp- | Formulation-1 | 0 | day | 50 | — |
| B as Adjuvant | | 42 | day | 18102 | 91 |
| Pnu Type-3-CRM CJ with Alum | Formulation-2 | 0 | day | 50 | — |
| as Adjuvant | | 42 | day | 9051 | 111 |
| Pnu Type-19A-CRM CJ with | Formulation-3 | 0 | day | 50 | — |
| rTbp-B as Adjuvant | | 42 | day | 2263 | 78 |
| Pnu Type-19A-CRM CJ with | Formulation-4 | 0 | day | 50 | — |
| Alum as Adjuvant | | 42 | day | 800 | 11 |
| Pnu Type-3-CRM CJ with fHbp | Formulation-5 | 0 | day | 50 | — |
| as Adjuvant | | 42 | day | 200 | 4 |
| Pnu Type-19A-CRM CJ with | Formulation-6 | 0 | day | 50 | — |
| fHbp as Adjuvant | | 42 | day | 566 | 8 |
| Pnu Type-3-CRM CJ with No | Formulation-7 | 0 | day | 50 | — |
| Adjuvant added | | 42 | day | 50 | 5 |
| Pnu Type-19A-CRM CJ with | Formulation-8 | 0 | day | 50 | — |
| No Adjuvant added | | 42 | day | 50 | 4 |
| rTbp-B -Alone | Formulation-9 | 0 | day | 400 | NA |
| | | 42 | day | 144815 | |
| fHbp Alone | Formulation-10 | 0 | day | 50 | |
| | | 42 | day | 141 | |
| Alum alone (Placebo without | Formulation-11 | 0 | day | 50 | 4 |
| antigen) | | 42 | day | 50 | 4 |

Pnu = pneumococcal polysaccharide;
CJ = pneumococcal polysaccharide-carrier protein conjugate;
fHbp: factor H binding protein

From Table-8b, it is seen that, when employed as an adjuvant with pneumococcal polysaccharide and CRM conjugates (Pnu type-3 & Pnu type-19A), transferrin binding protein-B (Tbp-B) produced excellent IgG titers in comparison to alum. When Tbp-B was employed as an adjuvant, there was a two-fold increase in IgG titers in Pnu type-3 conjugate when compared to alum. When used with Tbp-B as an adjuvant, the Pnu type-19A combination showed >2-fold titer as compared to alum as adjuvant. When neither adjuvant was included in the formulation, no notable immunological response was seen with any of the conjugates.

Further, it is seen from Table-8 that OPA studies also provided comparable result, with Pnu type-3 conjugate demonstrating a comparable OPA titer of 91 with Tbp-B as adjuvant versus 111 with alum employed as adjuvant. However, when Tbp-B was employed as an adjuvant in Pnu type-19A conjugate, the OPA titer was enhanced to 78 as opposed to 11 when alum was used as an adjuvant.

It is also seen that when Tbp-B was utilized as an adjuvant in the formulation of pneumococcal conjugates, there was an improvement in immunogenicity. For Pnu type-3 and Pnu type-19A, IgG titers were 18102 and 2263, respectively, whereas when alum was used as an adjuvant, titers of 9051 and 800 was observed.

fHbp was also used as a comparative adjuvant against Tbp-B, in which no immune response was observed in terms of IgG and OPA in all the formulations tested. The results obtained demonstrates the potential of Tbp-B obtained by the method of the present disclosure as an adjuvant in vaccine formulations.

It is seen that when used as an adjuvant with pneumococcal polysaccharide-carrier protein (CRM) conjugates (pneumococcal serotypes 3 & 19A), recombinant transferrin binding protein-B (r-Tbp-B) produced excellent IgG titers in comparison to alum (Alum used is Aluminum phosphate (Adju-Phos™ at a concentration of 125 ug/dose (125 ug is concentration of Al⁺³)). It is seen that when r-Tbp-B was employed as an adjuvant, there is at least two-fold increase in IgG titers in pneumococcal type-3 conjugate when compared to alum. Also, when used with r-Tbp-B as an adjuvant, the pneumococcal type-19A combination showed accelerated titers. When neither adjuvant was included in the formulation, no notable immunological response was seen with any of the conjugates.

### Transferrin Binding Protein as Carrier Protein:

Pneumococcal conjugates were prepared using polysaccharides from Streptococcus pneumoniae serotypes 6A, 19A, and 22F; Tbp-B was used as the carrier protein. Conjugation was carried out using CDAP activation with the remaining reaction parameters being similar to conventional conjugation, employing CRM as the carrier protein. IgG titer and OPA tests were performed on sera following a day 42 bleed.

The formulation detail and the result obtained is provided in Table-9

TABLE 9
Immunogenicity Studies

| | IgG titer | OPA titer |
|---|---|---|
| Pneumococcal conjugate formulation | Day 42 (GMT) | Day 42 (GMT) |
| Pnu-type 6A-Tbp-B CJ | 1131 | 64 |
| Pnu-type 6A-CRM CJ | 3200 | 91 |
| Pnu-type 19A-Tbp-B CJ | 4525 | 152 |
| Pnu-type 19A-CRM CJ | 2263 | 128 |
| Pnu-type 22F-Tbp-B CJ | 72408 | 1024 |
| Pnu-type 22F-TT CJ | 409600 | 1024 |

Pnu = pneumococcal polysaccharide;
CJ = pneumococcal polysaccharide-carrier protein conjugate

Table-9 includes the comparative data for IgG and OPA titers (Day-42) for pneumococcal polysaccharides from serotypes 6A, 19A, and 22F conjugated with Tbp-B as carrier protein versus CRM/TT as carrier protein in pneumococcal polysaccharide-protein conjugates.

IgG titers for Pnu-6A-Tbp-B & Pnu-CRM conjugates were found to be 1131 and 3200 respectively. Also, Pnu-19A conjugate showed a titer of 2263, and Pnu-19A-Tbp-B CJ have a titer of 4525 which was 2-fold as compared with CRM conjugates. Pnu-22F conjugated with TT showed higher titer than Tbp-B conjugated Pnu-22F. However median values for IgG titers are not yet established for Tbp-B conjugates, and any titer above the baseline titer of 50 (Day 0) shall be considered as immunogenic response.

OPA titers for all the Tbp-B conjugates were comparable with pneumococcal-CRM conjugates which revealed the formation of protective functional antibodies in both the cases (when Tbp-B was used as carrier protein, and also when CRM/TT were used as the carrier protein).

It is seen from above results that for Pnu Type 3-Tbp-B conjugate, the immune response increased by at least 2-fold (IgG) as compared to Pnu Type 3-CRM conjugate. Similarly, for Pnu Type 19A-Tbp-B conjugate, the immune response increases by at least 2-fold (IgG) and at least 7 fold (OPA) as compared to Pnu Type 19A-CRM conjugate.

### Studies on Adjuvant Activity of Recombinant Transferrin Binding Protein of the Present Disclosure

Studies carried out reveal that the recombinant transferrin binding protein when used as an adjuvant in a vaccine formulation was capable of slow release of antigens, which proves the adjuvanticity of the recombinant transferrin binding protein of the present disclosure.

It is known that Antigen-presenting cells (APCs), such as dendritic cells (DCs) are able to initiate innate immune responses with the help of Toll-like receptors (TLRs), which in turn trigger adaptive immunological reactions. Further, the saturated fatty acid (lipids) increase the production of Major histocompatibility complex (MHC) class II, cytokines (IL-12p70 and IL-6), and co-stimulatory molecules (CD40, CD80, and CD86) in bone marrow-derived DCs.

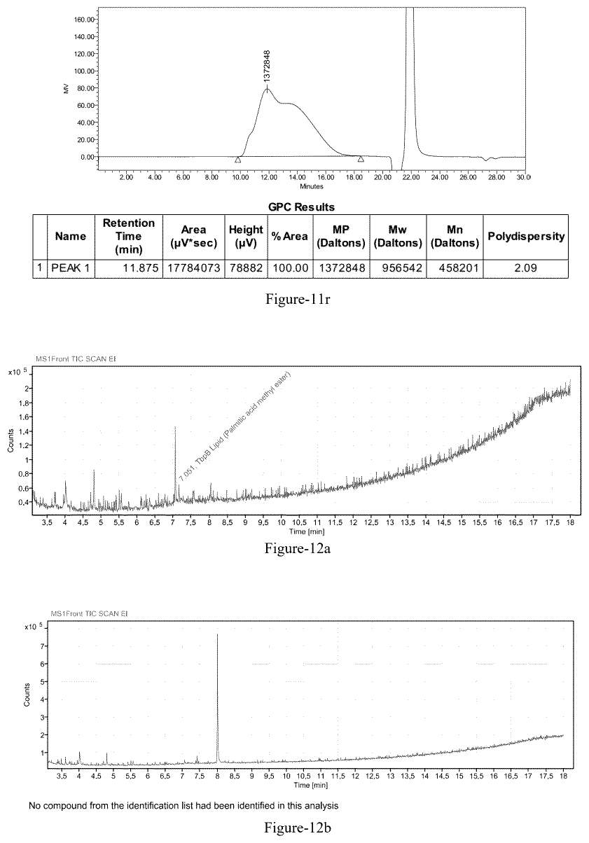

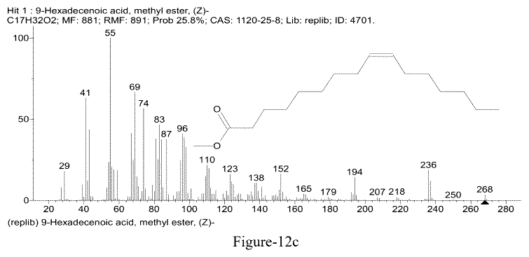

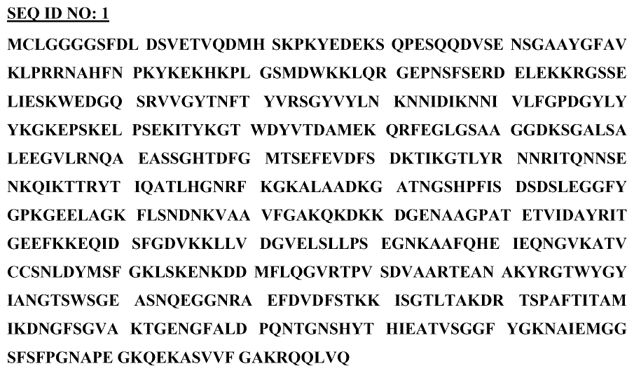

The analysis of the recombinant transferrin protein of the present disclosure revealed the presence of saturated palmitic acid (also known as hexadecanoic acid), a lipid molecule, and its existence was confirmed by gas chromatography-mass spectrometry (GC-MS) as illustrated in FIGS. 12a-12c. it is well established that lipoproteins are known immunogens and may act as adjuvants. When the lipid content of r-Tbp-B and CRM197 was analyzed, Hexadecanoic acid showed a clear peak at a retention time (RT) of 7.051 (FIG. 12a), whereas there was no such peak observed for CRM197 at that Retention Time (RT) (FIG. 12b). The peak was subsequently validated using NIST Library mass spectrometry, which showed that r-Tbp-B contained lipids but CRM197 did not contain such lipids (FIG. 12c). Thus, it is concluded that r-Tbp-B is a lipoprotein that has an adjuvant activity that activates/triggers immune response.

Applicant has found improved cost-effective methods (single chromatography step) of preparing highly purified & stable recombinant transferrin binding proteins (r-Tbp-B) and their use as adjuvant for vaccines.

Applicant provides here, an improved protocol circumventing some of the outlined issues raised above for the purification of recombinant r-Tbp-B from E. coli. The protocol utilizes a one-step ion exchange chromatography protocol (removing the need for affinity tags), said process utilizes sonication for extracting recombinant r-Tbp-B from E coli (instead of costly and non-compatible glass homogenizers) or said process is rapid, reduces the formation of aggregates, provides protein that retains its integrity, provides improved recovery with less impurity. Thus, to conquer the significant issues concerned with the production of r-Tbp-B, a simple, scalable, commercially viable and steadier process for high recovery of r-Tbp-B has been currently developed and disclosed in the present invention. The process of the present invention comprises streamlined, orthogonal, robust and scalable downstream process steps for production of r-Tbp-B at industrial scale with higher yield and purity.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps and can mean “includes”, “including”, and the like; “consisting essentially of” or “consists essentially” likewise is open-ended, allowing for the presence of more than that which is recited so long as basic or novel characteristics of that which is recited is not changed by the presence of more than that which is recited, but excludes prior art embodiments.

The use of the expression “one or more” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the composition of this invention, within the scope of the invention, may occur to those skilled in the art upon reviewing the disclosure herein. Such variations or modifications are well within the spirit of this disclosure.

The numerical values given for various physical parameters, dimensions and quantities are only approximate values and it is envisaged that the values higher than the numerical value assigned to the physical parameters, dimensions and quantities fall within the scope of the invention unless there is a statement in the specification to the contrary.

Similarly, the components used in purification, e.g., filters, columns, are not intended to be in any way limiting or exclusionary, and can be substituted for other components to achieve the same purpose at the discretion of the practitioner.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustration of the disclosure and not as a limitation.

### TECHNICAL ADVANTAGES

The method for obtaining purified transferrin binding protein of the present disclosure described herein above has several technical advantages including, but not limited to, the realization of:
A simple, scalable, commercially viable, cost-effective, and steadier process for obtaining r-Tbp-B with high recovery, low impurity/aggregate content (at least 95% purity, <5% impurity), retaining the integrity of protein structure;
Using Luria-Bertani Broth (LB) (having Yeast extract+Tryptone) media leading to OD at 590 nm values of 0.5 to 0.7 during IPTG induction, followed by OD values of 3.00±1.00 during harvest forming inclusion bodies of the recombinant rTbp-B;
Using single chromatography (Anion Exchange Chromatography) step method for obtaining high purity r-Tbp-B;
Avoids the use of tagged protein for obtaining purified r-Tbp-B;
Process utilizes sonication for extracting recombinant r-Tbp-B from E. coli (instead of costly and non-compatible glass homogenizers);
r-Tbp-B obtained is stable at 2-8° C., 25° C. & 37° C. (at least 90% purity retained on storage after 6 months);
Vaccine compositions containing r-Tbp-B is capable of inducing strong B and T cell responses;
r-Tbp-B slowly releases antigen, allowing antigen to be exposed to immune system for longer periods of time, resulting in higher B and T cell response;
r-Tbp-B when used as adjuvant for pneumococcal polysaccharide-protein conjugates, provides improved (at least 2-fold improved IgG) immune response as compared to Alum & non-inferior immune response (OPA) as compared to Alum;
r-Tbp-B as ideal novel platform adjuvant that can be used not only for conjugate vaccines, but also for other antigens. r-Tbp-B exhibits broad-spectrum of safety, shows either equivalent or improved immune response to vaccine antigens when compared to responses derived by using conventional Alum and previously known alternative adjuvants, is economical from manufacturing perspective, shows ease of production and use, is stable, is compatible with antigen (does not degrade antigen) and effectively activates humoral and cellular immune responses with no adverse reactions;
r-Tbp-B when used as carrier protein in pneumococcal polysaccharide-protein conjugates shows 2-fold increase in titre as compared with CRM conjugates;
The immune response in the subject (when rTbp-B is used) is increased by at least 2-fold (IgG Type 3-CRM conjugate) and at least 7-fold (OPA, as compared to Type 19A-CRM conjugate) and at least 2-fold (IgG, as compared to Type 19A-CRM conjugate).

1. A method for manufacturing a recombinant transferrin binding protein comprising a single chromatography step, said method comprising the following steps:
a. providing a cloning vector comprising a gene sequence encoding recombinant transferrin binding protein B;
b. transferring the cloning vector into a host system and growing the host system;
c. inducing the host system using an inducing agent at a final concentration in the range of 0.1 mM to 2.0 mM, preferably 0.1 mM to 1 mM;
d. harvesting/collecting the host system cells and subjecting it to centrifugation to collect the pellet containing inclusion bodies, wherein the transferrin binding protein is contained in the inclusion bodies;
e. washing the inclusion bodies, followed by solubilizing the inclusion bodies to obtain crude recombinant transferrin binding protein; and
f. subjecting the crude recombinant transferrin binding protein to an ion exchange chromatography to obtain the recombinant transferrin binding protein,
wherein the recombinant transferrin binding protein has >95% purity.

2. The method as claimed in claim 1, wherein the recombinant transferrin binding protein is obtained from a bacteria selected from the group comprising Streptococcus spp.; Salmonella spp.; Shigella spp.; E. coli; Neisseria meningitidis; Neisseria gonorrhoeae; Haemophilus influenzae; Haemophilus pneumonia; Helicobacter pylori; Chlamydia pneumoniae; Chlamydia trachomatis; Ureaplasma urealyticum; Mycoplasma pneumoniae; Staphylococcus spp.; Enterococcus faecalis; Enterococcus faecium; Bacillus anthracis; Vibrio cholerae; Pasteurella pestis; Pseudomonas aeruginosa; Campylobacter spp.; Mycobacterium spp.; Moraxella catarrhalis; Klebsiella pneumoniae; Treponema spp.; Borrelia spp.; Borrelia burgdorferi; Leptospira spp.; Hemophilus ducreyi; Corynebacterium diphtheria; Bordetella pertussis; Bordetella parapertussis; Bordetella bronchiseptica; Ehrlichia spp.; and Rickettsia spp, and
wherein Neisseria meningitidis is selected from the group consisting of serotypes A, B, B16, B6, C, D, E29, H, I, K, K454 L, M, W135, X, Y, and Z.

3. (canceled)

4. The method as claimed in claim 42, wherein the cloning vector is selected from the group comprising pET-30a (+), pET 23b, pET 28a, pET 28b, pET 23a, and pET 22a.

5. (canceled)

6. The method as claimed in claim 54, wherein the host system is selected from the group comprising Bacillus brevis, Bacillus megaterium, Bacillus subtilis, Caulobacter crescentus, Escherichia coli, Saccharomyces cerevisiae, Spodoptera frugiperda SF9, Spodoptera frugiperda SF21, baculovirus cells, Human Embryonic Kidney 293 cell line and Chinese Hamster Ovary cell line 1, and
wherein Escherichia coli is selected from the group consisting of E. coli BL21, E. coli BL21(DE3), E. coli BL21Star(DE3), E. coli Origami(DE3), BL21(DE3)pLysS, E. coli BL21-CodonPlus(DE3)-RIPL, E. coli Rosetta(DE3), E. coli C41(DE3)/C43(DE3), E. coli Lemo21(DE3); and E. coli Shuffle strain.

7. (canceled)

8. The method as claimed in claim 16, wherein the inducing agent is selected from lactose, isopropyl β-D-1-thiogalactopyranoside (IPTG), and functionally equivalent analogues.

9. The method as claimed in claim 18, wherein washing of inclusion bodies comprises the following steps:
a. adding a lysis buffer to the inclusion bodies;
b. adding 1 mg/ml to 10 mg/ml lysozyme, followed by incubation at 36° C. to 38° C., and agitation at 150 RPM to 300 RPM for a time period of 30 minutes to 180 minutes;
c. sonicating the solution at 50 Amp to 200 Amp for a time period of 1 minute to 10 minutes;
d. centrifuging the solution at 5000 RPM to 20000 RPM for 5 minutes to 20 minutes at 2° C. to 8° C.;
e. decanting the supernatant and collecting the pellet;
f. adding wash buffer to the pellet, mixing thoroughly and centrifuging the solution at 5000 RPM to 20000 RPM for 5 minutes to 20 minutes at 2° C. to 8° C.;
g. decanting the supernatant and collecting the pellet;
h. adding 0.1 M to 2 M NaCl to the pellet, mixing thoroughly and centrifuging the solution at 5000 RPM to 20000 RPM for 5 minutes to 20 minutes at 2° C. to 8° C.;
i. decanting the supernatant and collecting the pellet;
j. adding distilled water to the pellet, mixing thoroughly and centrifuging the solution at 5000 RPM to 20000 RPM for 5 minutes to 20 minutes at 2° C. to 8° C.;
k. decanting the supernatant and collecting the pellet;
l. washing the pellet with distilled water/Milli-Q, mixing thoroughly with distilled water/Milli-Q water and centrifuging the solution at 5000 RPM to 20000 RPM for 5 minutes to 20 minutes at 2° C. to 8° C.;
m. adding Tris-Cl buffer and incubating at 36° C. to 38° C. for a time period of 30 minutes to 180 minutes; and
n. centrifuging the solution at 5000 RPM to 20000 RPM for 5 minutes to 30 minutes at 2° C. to 8° C. to separate the supernatant and the pellet.

10. The method as claimed in claim 9, wherein
the lysis buffer comprises 10 mM to 100 mM Tris-Cl, 10 mM to 100 mM EDTA, 5% to 50% sucrose, and pH 7.0 to 9.0; and
the wash buffer comprises 0.1 M to 2 M NaCl, 0.5% to 10% Triton X-100.

11. The method as claimed in claim 10, wherein the inclusion bodies are solubilized using a sonication buffer and a dispersion buffer,
wherein the sonication buffer comprises 50 mM to 200 mM Tris-C1, 10 mM to 100 mM glycine and pH in the range of 7.0 to 9.0, and
wherein the dispersion buffer comprises 20 mM to 200 mM Tris-C1, 10 mM to 100 mM glycine, 5 M to 15 M urea, preferably 4 M to 8M, and pH in the range of 7.0 to 9.0.

12. (canceled)

13. (canceled)

14. The method as claimed in claim 11, wherein the ion exchange chromatography is anion exchange chromatography and the resin is selected from the group comprising methacrylate polymer; sepharose; diethylaminoethyl cellulose; crosslinked agarose beads; ceramic beads filled with a functionalized hydrophilic gel; methacrylate with trimethylammoniumethyl ligands; highly cross-linked cellulose with dextran scaffold; polystyrene/divinyl benzene polymer matrix; agarose matrix; hydrophilic polyvinyl ether base matrix; hydroxylated methacrylic polymer; sephadex base matrix; and hydrophilic polymeric support.

15. The method as claimed in claim 14, wherein the single step chromatography is carried out by a multimodal resin having anion exchange.

16. The method as claimed in claim 15, wherein the crude transferrin binding protein is pre-treated before being subjected to chromatography, and
wherein the pre-treatment is carried out by dialysis using a buffer comprising 10 mM to 100 mM Tris, and 50 mM to 200 mM NaCl.

17. (canceled)

18. A recombinant transferrin binding protein obtained by claim 1 is at least 90% identical to SEQ ID NO: 1.

19. The recombinant transferrin binding protein as claimed in claim 18, wherein the molecular weight of the recombinant transferrin binding protein is in the range of 20 kDa to 100 kDa (SEC-MALS).

20. A vaccine composition comprising the recombinant transferrin binding protein obtained by the method as claimed in claim 16, wherein the concentration of the recombinant transferrin binding protein in the vaccine composition is in the range of 1 μg to 100 μg.

21. The vaccine composition as claimed in claim 20, wherein the composition comprises:
a. at least one polysaccharide-carrier protein conjugate;
b. recombinant transferrin binding protein as an adjuvant or a carrier protein; and
c. pharmaceutically acceptable excipients, or
wherein the composition comprises
d. recombinant transferrin binding protein as an antigen; and
e. pharmaceutically acceptable excipients.

22. (canceled)

23. The vaccine composition as claimed in claim 21, wherein the polysaccharide is derived from a bacteria selected from the group comprising Streptococcus spp, Salmonella spp., Shigella spp., E. coli, Neisseria meningitidis, Neisseria gonorrhoeae, Haemophilus influenzae, Haemophilus pneumonia, Helicobacter pylori, Chlamydia pneumoniae, Chlamydia trachomatis, Ureaplasma urealyticum, Mycoplasma pneumoniae, Staphylococcus spp., Enterococcus faecalis, Enterococcus faecium, Bacillus anthracis, Vibrio cholerae, Pasteurella pestis, Pseudomonas aeruginosa, Campylobacter spp., Clostridium spp., Mycobacterium spp., Moraxella catarrhalis, Klebsiella pneumoniae, Treponema spp., Borrelia spp., Borrelia burgdorferi, Leptospira spp., Hemophilus ducreyi, Corynebacterium diphtheria, Bordetella pertussis, Bordetella parapertussis, Bordetella bronchiseptica, Ehrlichia spp., and Rickettsia spp,
wherein a Streptococcus spp. selected from the group comprising Group A Streptococcus, Group B Streptococcus selected from group Ia, Ib, II, III, IV, V, VI, VII, VII, VIII, and IX, Streptococcus pneumoniae, Streptococcus pyogenes, Streptococcus agalactiae, and Streptococcus viridans,
wherein a Streptococcus pneumoniae serotype selected from the group consisting of 1, 2, 3, 4, 5, 6A, 6B, 6C, 6D, 6E, 6G, 6H, 7A, 7B, 7C, 7F, 8, 9A, 9L, 9F, 9N, 9V, 10F, 10B, 10C, 10A, 11A, 11F, 11B, 11C, 11D, 11E, 12A, 12B, 12F, 13, 14, 15A, 15C, 15B, 15F, 16A, 16F, 17A, 17F, 18C, 18F, 18A, 18B, 19A, 19B, 19C, 19F, 20, 20A, 20B, 21, 22A, 22F, 23A, 23B, 23F, 24A, 24B, 24F, 25F, 25A, 27, 28F, 28A, 29, 31, 32A, 32F, 33A, 33C, 33D, 33E, 33F, 33B, 34, 45, 38, 35A, 35B, 35C, 35F, 36, 37, 38, 39, 40, 41F, 41A, 42, 43, 44, 45, 46, 47F, 47A, and 48,
wherein a Salmonella spp. is selected from the group comprising Salmonella typhi, Salmonella paratyphi, Salmonella enteritidis, and Salmonella typhimurium,
wherein a Neisseria meningitidis serotype is selected from the group consisting of A, B, C, E29, H, I, K, L, M, W135, X, Y, and Z, and
wherein a Staphylococcus spp. is selected from the group comprising Staphylococcus aureus, Staphylococcus aureus type 5, and Staphylococcus aureus type 8.

24. (canceled)

25. (canceled)

26. The vaccine composition as claimed in claim 21, wherein the pharmaceutically acceptable excipient is selected from the group comprising stabilizers, pharmaceutically acceptable transporter, binder, carrier, isotonic agent, emulsifier, humectant, surfactants, polymers, preservatives, diluent, and salts.

27. The vaccine composition as claimed in claim 21, wherein the composition elicits at least 2-fold higher IgG titers when recombinant transferrin binding protein is present in the composition, or
wherein Pnu Type 3-Tbp-B conjugate shows increased immune response by at least 2-fold (IgG) as compared to Pnu Type 3-CRM conjugate; and Pnu Type 19A-Tbp-B conjugate shows increased immune response by at least 2-fold (IgG) and at least 7-fold (OPA) as compared to Pnu Type 19A-CRM conjugate.

28. (canceled)

29. The vaccine composition as claimed in claim 21, wherein the composition is administered via a route selected from oral, intranasal, intramuscular, intradermal, subcutaneous and intravenous.

30. A method for preventing or treating a bacterial infection in a subject, comprising administering to a subject a vaccine composition comprising a recombinant transferrin binding protein obtained by the method as claimed in claim 1.


SEQUENCE LISTING (1 sequence)

SEQ ID NO: 1 - protein, 580 aa, Synthetic construct
MCLGGGGSFDLDSVETVQDMHSKPKYEDEKSQPESQQDVSENSGAAYGFAVKLPRRNAHFNPKYKEKHKPLGSMDWKKLQRGEPNSFSERDELEKKRGSSELIESKWEDGQSRVVGYTNFTYVRSGYVYLNKNNIDIKNNIVLFGPDGYLYYKGKEPSKELPSEKITYKGTWDYVTDAMEKQRFEGLGSAAGGDKSGALSALEEGVLRNQAEASSGHTDFGMTSEFEVDFSDKTIKGTLYRNNRITQNNSENKQIKTTRYTIQATLHGNRFKGKALAADKGATNGSHPFISDSDSLEGGFYGPKGEELAGKFLSNDNKVAAVFGAKQKDKKDGENAAGPATETVIDAYRITGEEFKKEQIDSFGDVKKLLVDGVELSLLPSEGNKAAFQHEIEQNGVKATVCCSNLDYMSFGKLSKENKDDMFLQGVRTPVSDVAARTEANAKYRGTWYGYIANGTSWSGEASNQEGGNRAEFDVDFSTKKISGTLTAKDRTSPAFTITAMIKDNGFSGVAKTGENGFALDPQNTGNSHYTHIEATVSGGFYGKNAIEMGGSFSFPGNAPEGKQEKASVVFGAKRQQLVQ